(19) United States
(12) Patent Application Publication
(10) Pub. No.: US 2026/0364138 A1
(43) Pub. Date: Nov. 19, 2026

(54) ENRICHED ASPARAGUS RACEMOSUS ROOT COMPOSITION AND PROCESS FOR PREPARATION THEREOF

(71) Applicant: LAILA NUTRA PRIVATE LIMITED, Vijayawada, Andhra Pradesh (IN)

(72) Inventors: Ganga Raju GOKARAJU, Vijayawada, Andhra Pradesh (IN); Rama Raju GOKARAJU, Vijayawada, Andhra Pradesh (IN); Kiran BHUPATHIRAJU, Vijayawada, Andhra Pradesh (IN); Krishanu SENGUPTHA, Vijayawada, Andhra Pradesh (IN); Venkateswarlu SOMEPALLI, Vijayawada, Andhra Pradesh (IN); Venkata Kanaka Ranga Raju GOKARAJU, Vijayawada, Andhra Pradesh (IN); Trimurtulu GOLAKOTI, Vijayawada, Andhra Pradesh (IN); Venkata Krishna Raju ALLURI, Vijayawada, Andhra Pradesh (IN)

(21) Appl. No.: 19/120,581

(22) PCT Filed: May 15, 2024
(86) PCT No.: PCT/IN2024/050563

§ 371 Date: Apr. 11, 2025

(30) Foreign Application Priority Data
May 25, 2023 (IN) 202341036296

(51) Int. Cl. A61K 36/8965; A61K 9/16; A61K 31/7048; A61P 15/08; C07J 71/00

(52) U.S. Cl. CPC A61K 36/8965; A61K 9/1611; A61K 9/1652; A61K 31/7048; A61P 15/08; C07J 71/0005; A61K 2236/331; A61K 2236/333; A61K 2236/53

(57) ABSTRACT

The present invention discloses synergistic enriched Asparagus racemosus root extract composition comprising; shatavarin I, dehydroshatavarin I, shatavarin IV, and shatavarin IX; process for the preparation; methods of treatment and use of the compositions for female health benefits.

### TECHNICAL FIELD OF THE INVENTION

The present invention relates to a synergistic enriched Asparagus racemosus root composition comprising; (i) shatavarin I; (ii) dehydroshatavarin I; (iii) shatavarin IV and (iv) shatavarin IX; process for preparing the same and method of use thereof for female health benefits.

### BACKGROUND OF THE INVENTION

Hormones are chemical messengers produced by various endocrine glands of the body that are required for the maintenance of various metabolic processes. An imbalance occurs when there is too much or too little of a hormone, which can disrupt numerous aspects of an individual's life. Women are more prone to such imbalances compared to men. Variation in hormone secretions is more prominent during puberty, perimenopause, and menopause. Several factors, including medications, chemotherapy, thyroiditis, and environmental pollutants, contribute to hormonal imbalances that can lead to infertility, premenstrual syndrome (PMS), polycystic ovary syndrome (PCOS), and polycystic ovary disease (PCOD) in women. The symptoms of hormonal imbalances in women include irregular periods, heavy periods, acne, weight gain, hirsutism (excessive facial hair growth), eating disorders, stress, anxiety, diabetes, mood swings, fatigue, and irritability. Pharmacological treatment options for women with hormonal imbalance include hormone replacement medications (to reduce hot flashes, and night sweats), anti-androgen medications, letrozole (to stimulate ovulation in PCOS condition), eflornithine (to slow excessive facial hair growth) etc. However, most of these treatment options for prolonged periods have several side effects on the body. Hence, the demand for alternative approaches such as vitamins, nutrients, minerals, and herbal remedies is growing as they are effective with fewer side effects.

Hormone therapies such as estrogen therapy, progesterone therapy or estrogen-progesterone combinations have proven to be most effective. However, there are definite risks to hormone therapy such as increased risk of breast cancer, stroke and heart disease.

Indian patent publication 202121017695 discloses an oral dosage form of shatavari comprising a mixture of alcoholic and aqueous extracts of shatavari (Asparagus racemosus) root uniformly impregnated on a sugar globule, wherein the concentration of active constituent shatavarin IV is 0.14-0.18%.

Another patent publication US2014303258A1 discloses a method of solubilizing water-insoluble bioactive compounds and metabolites thereof using natural triterpene glycosides as solubilizing agents. Shatavari glycosides obtained from Asparagus racemosus wherein shatavari glycosides are standardized for not less than 4% w/w (HPLC) shatavarin IV

US patent publication U.S. Pat. No. 6,649,745B2 discloses oligospirostanoside of formula 1, 3-0-[α-L-rhamnopyranosyl-(1→2)-α-L-rhamnopyranosyl-(1→4)-0-β-D-gluco pyranosyl]-25(S)-5β-spirostan-3β-ol isolated from Asparagus racemosus and a pharmaceutical composition comprising an effective amount of said oligospirostanoside 3-0-[α-L-rhamnopyranosyl-(1→2)-α-L-rhamnopyranosyl-(1Δ4)-0-β-D-glucopyranosyl]-25(S)-5β-spirostan-3β-ol contained in a pharmaceutically acceptable carrier.

Non-patent literature Churanya Onloma et al (Natural Product Communications, 2017; Vol. 12 (1)) discloses a method for quantitative analysis of saponin glycosides bioactive constituents in Asparagus racemosus. A high-performance liquid chromatography quadrupole time of flight mass spectrometry (HPLC-Q-TOF-MS/MS) method was developed and validated for simultaneous determination of five saponin glycosides, asparacoside, shatavarin IX, shatavarin IV, asparanin A, and shatavarin V in A. racemosus extracted with 70% MeOH.

However, there are no extract which report a product of Asparagus racemosus roots with enriched active phytochemicals. Thus, there remains a need in the art to provide an improved product of Asparagus racemosus roots, with enriched active phytochemicals thereby, increasing its efficacy in treating issues pertaining to female health with reduced side effects.

### Objective of the Invention

The primary objective of the present invention is to provide enriched Asparagus racemosus root composition comprising; (i) shatavarin I (ii) dehydroshatavarin I (iii) shatavarin IV, and (iv) shatavarin IX; for female health benefits and process for preparation thereof.

Another objective of the present invention is to provide formulations of the enriched Asparagus racemosus root composition and process for preparation of the same.

Further objective of the invention is to provide a method of treatment and use of the enriched Asparagus racemosus root composition for female health benefits.

### SUMMARY OF THE INVENTION

Accordingly, in one aspect, the present invention provides synergistic enriched Asparagus racemosus root extract comprising; (i) shatavarin I (ii) dehydroshatavarin I (iii) shatavarin IV and (iv) shatavarin IX; for female health benefits.

In another aspect, the present disclosure provides synergistic enriched Asparagus racemosus root extract comprising; (i) shatavarin I (ii) dehydroshatavarin I (iii) shatavarin IV and (iv) shatavarin IX; wherein the shatavarin I in the range of 0.5-15%; dehydroshatavarin I in the range of 0.1-25%; shatavarin IV in the range of 2-25% and shatavarin IX in the range of 0.001-1.0%; for female health benefits.

In yet another aspect, the present invention provides a process for the preparation of synergistic enriched Asparagus racemosus root extract comprising; (i) shatavarin I (ii) dehydroshatavarin I (iii) shatavarin IV (iv) shatavarin IX comprising the steps of (a) enriching water extract of Asparagus racemosus (Shatavari) root by re-extracting with a polar organic solvent or by loading water extract on macroporous resin and eluting with an aqueous alcohol to obtain enriched extract; (b) extracting spent or residual Asparagus racemosus (Shatavari) root with polar organic solvent to obtain second extract; (c) combining the extracts obtained from step (a) and step (b) to obtain enriched Asparagus racemosus composition; and d) optionally blending the enriched Asparagus racemosus composition with at least one component selected from pharmaceutically or nutraceutically or dietically acceptable excipients, carriers and diluents.

In another aspect, the present disclosure provides composition/formulations of the synergistic enriched Asparagus racemosus root extract comprising; (i) shatavarin I (ii) dehydroshatavarin I (iii) shatavarin IV (iv) shatavarin IX and at least one component selected from pharmaceutically or nutraceutically or dietically acceptable excipients, carriers, and diluents.

In another aspect, the present invention provides a process for preparation of the composition of the synergistic enriched Asparagus racemosus root extract comprising; (i) shatavarin I (ii) dehydroshatavarin I (iii) shatavarin IV (iv) shatavarin IX comprising a step of blending with at least one component selected from pharmaceutically or nutraceutically or dietically acceptable excipients, carriers, and diluents.

Yet, another aspect of the present disclosure provides a method of obtaining at least one female reproductive and sexual health benefit selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, menstrual cramps; alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation; wherein the method comprises supplementing the human subject in need thereof with an effective dose of a synergistic enriched Asparagus racemosus root extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and optionally with at least one component selected from pharmaceutically or nutraceutically or dietically acceptable excipients, carriers, and diluents.

Yet, another aspect of the invention provides use of the present synergistic enriched Asparagus racemosus root extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and optionally with at least one component selected from pharmaceutically or nutraceutically or dietically acceptable excipients, carriers, and diluents; for obtaining at least one female reproductive and sexual health benefits selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, menstrual cramps; alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation.

In a further aspect, the invention provides hitherto unreported novel shatavarin, i.e., dehydroshatavarin I of the formula;

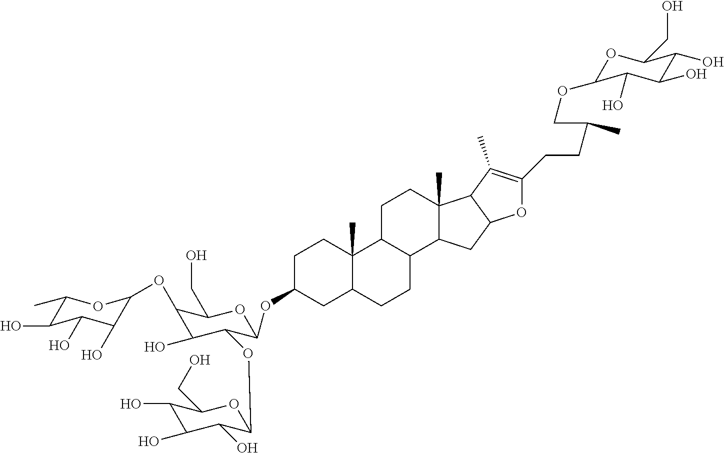

Characterized by ¹H NMR (d₆-DMSO): δ 5.16 (1H, d, J=4.4 Hz), 5.13 (1H, d, J=4.0 Hz), 4.90 (1H, d, J=4.8 Hz), 4.81-4.85 (4H, m), 4.72 (1H, s), 4.62-4.68 (4H, m), 4.59 (1H, d, J=5.2 Hz), 4.47 (1H, d, J=8.0 Hz), 4.41 (1H, d, J=5.6 Hz), 4.37 (1H, t, J=6.4 Hz), 4.33 (1H, d, J=7.6 Hz), 4.27-4.32 (1H, m), 4.16 (1H, t, J=5.6 Hz), 4.08 (1H, d, J=8.0 Hz), 3.64-3.67 (3H, m), 3.61 (1H, brs), 3.57-3.58 (1H, m), 3.37-3.51 (7H, m), 3.03-3.19 (9H, m), 2.92-3.01 (2H, m), 2.01-2.13 (3H, m), 1.63-1.84 (5H, m), 1.55 (3H, s), 1.36-1.52 (9H, m), 1.15-1.28 (5H, m), 1.10 (3H, d, J=6.4 Hz), 1.06-1.09 (2H, m), 0.95-1.05 (2H, m), 0.90 (3H, s), 0.89 (3H, d, J=6.4 Hz), 0.62 (3H, s). Mass (Q-tof, negative ion mode): m/z 1047.5400 (M−H)⁻.

### BRIEF DESCRIPTION OF THE DRAWING

FIG. 1 illustrates the Chemical structures of shatavarins isolated from Asparagus racemosus root.

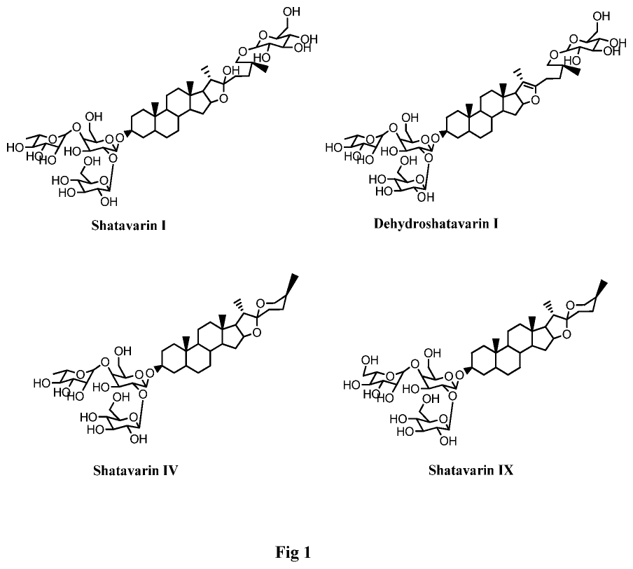

FIG. 2: HPLC chromatogram of Asparagus racemosus enriched extract composition.

### DETAILED DESCRIPTION OF THE INVENTION

The invention will now be described in detail in connection with certain preferred and optional embodiments so that various aspects thereof may be more fully understood and appreciated.

Unless specified otherwise, all technical and scientific terms, such as but not limited to chemical structures, process steps, or materials disclosed herein, are intended, but also include equivalents thereof as commonly understood by those ordinarily skilled in the art to which this invention belongs. To describe the invention, certain terms are defined herein specifically as follows.

The term “enriched Asparagus racemosus root extract” or “enriched Asparagus racemosus root extract composition” or “enriched Asparagus racemosus composition” or “synergistic enriched Asparagus racemosus root extract composition” as referred alternately herein throughout the specification infers and includes an Asparagus racemosus root extract comprising (i) shatavarin I (ii) dehydroshatavarin I (iii) shatavarin IV and (iv) shatavarin IX.

The chemical compounds or simply compounds may be identified either by their chemical structure, chemical name or common name as used herein. The compounds used herein may contain one or more chiral centres and/or double bonds and, therefore may exist as isomers. The term “methods of obtaining female health function” or “improving” refers to inhibiting, preventing, or arresting the development of pathology (disease, disorder, or condition) and/or causing the reduction, remission, or regression of a pathology. Those with skill in the art will understand that various methodologies and assays can be used to assess the development of pathology, and similarly, various methodologies and assays may be used to assess the reduction, remission, or regression of pathology.

### Geographical Origin

The source of the herb used in the invention is as follows:

Asparagus racemosus root was procured from a vendor located in Mumbai, Maharashtra, India.

Asparagus racemosus: It is also called as shatavari or satavari or satavar belonging to the family Asparagaceae. It is known as the “Queen of Herbs” and it is one of the prominent medicinal herbs in Ayurveda. It is considered as a hormone balancer and a general tonic to uplift male as well as female health and vitality. Its medicinal value has been reported in Indian traditional medicine such as Ayurveda, Unani, and Siddha. “Shatavari is highly effective in problems related to the female reproductive system. The ancient writing “Charak Samhita” mentions the use of Asparagus racemosus to treat disorders related to women's health. In modern Ayurvedic practices, the roots of the plant is considered to be effective as an anti-spasmodic, appetizer, stomach tonic, aphrodisiac, galactogogue, astringent, anti-diarrheal, anti-dysenteric, laxative, anti-cancer, anti-inflammatory, blood purifier, anti-tubercular, anti-epileptic and also in alleviating night blindness, kidney problems, jaundice and throat complaints. Furthermore, it is also known as “Medhya”—the plants which increase intelligence and promote learning and memory. Being a potent immunity stimulator, Shatavari increases the body's resistance during normal and immune-suppressed conditions, helping to boost immunity during diseased conditions and aiding in the recovery of the immune system. Pharmacologically the extract is reported as anti-oxidant, anti-stress antiulcer, wound healing, antitussive, gastroprotective, neuroprotective etc.

Asparagus racemosus extracts have various extractions process, for example, water extract and alcohol extracts, and exist in the market with standardization of total saponins by the gravinetric method of analysis. In the scientific literature, shatavarins I-X were isolated, and the chemical structures were characterized by spectroscopic analysis (In one article, methanol extract was standardized for shatavarin IV, as shown in Table-1. However, there is no report on the standardization of extracts for multiple phytochemical markers present in Asparagus racemosus especially dehydroshatavarin I or the compositions containing dehydroshatavarin I.

TABLE 1
Content of markers in methanol extract reported in the literature

| Extraction | Shatavarin | Shatavarin | Shatavarin | |
|---|---|---|---|---|
| method | I | IV | IX | Reference |
| Methanol | Not | 0.05 to 0.4% | Not | Sagnik Haldar, et al., |
| extract | estimated | | estimated | Journal of Planar |
| | | | | Chromatography, |
| | | | | 2018, 3, 197-20 |

Generally, shatavari root was extracted with water or aq. ethanol for industrial and other research purposes. But these solvents do not extract the total shatavarins (active constituents) that are present in the roots. So, a more general process is needed for total extraction of active constituents.

Hence, the inventors conducted several experiments and surprisingly found a novel two-step extraction process that gave total active phytochemicals in optimum concentrations. In one of the embodiments, the process begins with the maceration of raw material with water for 16 h, and then spent raw material after water extraction was again extracted with 90% aq. ethanol to obtain spent 90% ethanol extract (AR-4). The water extract was subjected to enrichment with respect to total shatavarin compounds using one of the two following processes, 1) re-extraction of water extract with 90% aq. Ethanol to obtain the enriched extract (AR-3), 2) alternatively, the water extract was processed over macroporous resin or subject to membrane filtration to obtain the enriched extract (AR-11) or subjected to membrane filtration (AR-12). In the final step, the spent 90% ethanol extract (AR-4) was combined with one of the enriched water extracts (AR-3 or AR-11 or AR-12) to obtain the novel shatavari extract composition containing the total active phytochemicals. Similarly, the inventors demonstrated the invention using various suitable polar solvents such as ethanol, n-butanol, methanol, acetone, aqueous ethanol, aqueous methanol, aqueous n-butanol, aqueous acetone and mixtures thereof and aqueous alcohols such as aqueous methanol, aqueous ethanol and aqueous n-butanol, for obtaining novel shatavari extract compositions containing the total active phytochemicals, as demonstrated under the examples.

Enrichment through macroporous resins: Macroporous resin refers to a type of polymer material that has the ability to adsorb or capture selected substances onto its surface. It is commonly used in various industries for purification, separation, and removal of specific molecules or ions from a mixture. They are widely used in the extraction, purification, and enrichment of natural compounds from plant extracts, herbal medicines, and other natural sources. Adsorption of target compounds onto macroporous (adsorbent) resins from the crude extracts followed by their elution using organic solvent has proved to be an effective and efficient strategy for the purification of some natural products.

Thus, water extract of Asparagus racemosus was passed over the macroporous resin to bind the total shatavarins, washing the resin with water and then finally eluting the column with aqueous organic solvents such as aqueous ethanol and evaporating the solvent to give enriched extracts. The spent raw material after water extraction was again extracted with polar organic solvents (examples 8-11).

The inventive enriched Asparagus racemosus extract compositions were then analysed using isolated compounds by the analytical HPLC method to obtain the enriched Asparagus racemosus extract compositions with novel standardization, and the results were presented in Table-2 below.

TABLE 2
Estimation of four shatavarins in extracts obtained as per the process in examples
1 and 8 (each from 100 g of raw material) by HPLC method of analysis.

| | HPLC | | | | | |
|---|---|---|---|---|---|---|
| Process as in | Shatavarin | Dehydroshatavarin | Shatavarin | Shatavarin | | Wt. of 4 |
| Example≠ | I | I | IV | IX | Total | Shatavarins |
| Comp ex 1 | 1.96% | 0.19% | 0.50% | 0.11% | 2.76% | 1.24 g |
| (Water extract) | | | | | | |
| Comp ex 2 | 2.19% | 0.26% | 0.53% | 0.35% | 3.33% | 1.40 g |
| (90% aq. | | | | | | |
| ethanol extract) | | | | | | |
| Example-1 | 0.83% | 3.87% | 3.93% | 0.18% | 8.81% | 2.48 g |
| Example-8 | 4.93% | 10.00% | 13.20% | 0.04% | 28.17% | 2.86 g |

The process of the present invention results in higher yield of total four Shatavarins (2.48 g and 2.86 g, as per process in example-1 and example-8 respectively, compared to 1.24 g, 1.40 g, for water and aq. ethanol extracts individually). So, the new two-step process (both example-1 and example-8) considerably increases the total shatavarins recovery. Other solvents, such as butanol, methanol, and acetone were also used in place of 90% aq. ethanol to yield enriched Asparagus racemosus root extract (examples 2-7, 9-10). For comparison, water extract and 90% aq. ethanol extracts were also prepared individually (comparative examples 1-2).

In addition to increasing the yield of a total four shatavarins, the new two-step extraction process also significantly increases the dehydroshatavarin I, and shatavarin IV content in the extract. Dehydroshatavarin I was observed as entirely a new compound isolated from Shatavari root which was never isolated either from this species or other herbs. Shatavarin IV is one of the important active constituents of shatavari root. In example-1 and example-8, the shatavarin IV content of the extract is 1.1 g and 1.3 g, respectively, compared to 225 mg and 226 mg in water and aq. ethanol extracts individually. The two-step process (both example-1 and example-8) of the present invention unexpectedly increased the shatavarin IV recovery.

In an embodiment, the present invention discloses a novel two-step extraction process for Shatavari root that involves sequential extraction using both water and hydro alcohol. This process results in not only a higher yield of extract in terms of active constituents but also a broader range of active shatavarins, making the present extract more potent and efficacious than the extracts produced using traditional extraction methods.

Advantages of the present process over the prior art process:
Higher yield of total shatavarins.
The new compound dehydroshatavarin-I was isolated and enriched.
Higher shatavarin IV content.

In another embodiment, the present invention discloses enriched Asparagus racemosus extracts comprising; (i) shatavarin I; (ii) dehydroshatavarin I; (iii) shatavarin IV and (iv) shatavarin IX; for female health benefits.

In one preferred embodiment, the present invention provides enriched Asparagus racemosus extracts comprising; (i) shatavarin I; (ii) dehydroshatavarin I; (iii) shatavarin IV and (iv) shatavarin IX; wherein the shatavarin I is in the range of 0.5-15%; dehydroshatavarin I in the range of 0.1-25%; shatavarin IV in the range of 2-25% and shatavarin IX in the range of 0.001-1.0%.

In another embodiment, the present invention provides enriched Asparagus racemosus composition comprising; (i) shatavarin I; (ii) dehydroshatavarin I; (iii) shatavarin IV and (iv) shatavarin IX; (v) with at least one component selected from pharmaceutically or nutraceutically or dietically acceptable excipients, carriers, and diluents.

In another embodiment, the present invention provides enriched Asparagus racemosus composition comprising; (i) shatavarin I; (ii) dehydroshatavarin I; (iii) shatavarin IV and (iv) shatavarin IX; (v) with at least one component selected from pharmaceutically or nutraceutically or dietically acceptable excipients, carriers, and diluents; wherein the Asparagus racemosus composition varies in the range of 90%-10% by weight and excipients in the composition varies in the range of 10%-90% by weight.

In yet another embodiment, the present invention provides enriched Asparagus racemosus composition comprising; (i) shatavarin I; (ii) dehydroshatavarin I; (iii) shatavarin IV and (iv) shatavarin IX; wherein the shatavarin I is in the range of 0.5-15%; dehydroshatavarin I in the range of 0.1-25%; shatavarin IV in the range of 225% and shatavarin IX in the range of 0.001-1.0% and with at least one component selected from pharmaceutically or nutraceutically or dietically acceptable excipients, carriers, and diluents; for female health benefits.

In yet another embodiment, the present invention provides enriched Asparagus racemosus composition, wherein the composition is also standardized to total saponins by gravimetric method of analysis in the range of 20-90%.

In another embodiment, the present invention provides enriched Asparagus racemosus composition comprising; (i) shatavarin I; (ii) dehydroshatavarin I; (iii) shatavarin IV and (iv) shatavarin IX; optionally containing at least one additional ingredient selected from shatavarin II, shatavarin III, shatavarin V, shatavarin VI, shatavarin VII, shatavarin VIII, shatavarin IX, asparacoside, asparanin and immunoside or mixtures thereof.

In another embodiment, the present invention provides enriched Asparagus racemosus composition comprising; (i) shatavarin I; (ii) dehydroshatavarin I; (iii) shatavarin IV and (iv) shatavarin IX; optionally containing at least one extract or fraction selected from Nigella sativa, Cinnamomomum zeylanicum, Foeniculum vulgare, Trigonella foenum-graecum, Glycyrrhiza glabra and Linum usitatissimum or atleast one phytochemical selected from Inositols such as myo-inositol or D-chiro-inositol or mixtures thereof In yet another embodiment, the present invention provides enriched Asparagus racemosus compositions as described above; wherein the Asparagus racemosus extracts obtained from at least one plant part selected from the group comprising roots, leaves, stems, tender stems, tender twigs, aerial parts, whole fruit, fruit peel, fruit rind, seeds, flower heads, bark, hardwood, rhizome or whole plant or mixtures thereof.

In another embodiment, the present invention provides a process for the preparation of enriched Asparagus racemosus extract comprising; (i) shatavarin I; (ii) dehydroshatavarin I; (iii) shatavarin IV and (iv) shatavarin IX; comprising;
(a) extracting Asparagus racemosus root powder with water,
(b) evaporating the water extract to a minimum volume to obtain the concentrate,
(c) re-extracting the step (b) concentrate with a polar organic solvent,
(d) evaporating the polar organic solvent soluble extract to give an enriched extract,
(e) alternately passing the water extract of step (a) on adsorbent resin and eluting with aqueous alcohol,
(f) evaporating the aqueous alcohol eluent to give an enriched extract,
(g) extracting the Asparagus racemosus root spent residue from step (a) with a polar organic solvent,
(h) evaporating the polar organic solvent extract in step (g) to give a second extract,
(i) combining the extracts of step (d) or step (f) and step (h) to obtain enriched Asparagus racemosus composition;

The polar organic solvent used in the process for the preparation of enriched Asparagus racemosus extract composition is selected from but not limited to n-butanol, ethanol, methanol, aqueous ethanol, aqueous methanol, aqueous n-butanol and mixtures thereof.

In another embodiment, the present invention provides an alternative process for the preparation of enriched Asparagus racemosus composition comprising; (i) shatavarin I; (ii) dehydroshatavarin I; (iii) shatavarin IV and (iv) shatavarin IX; through loading the water extract of Asparagus racemosus onto to macroporous resin column and eluting the column through aqueous organic solvents such as aqueous ethanol and combining the fractions.

In another embodiment, the present invention provides a process for preparation of the composition of the enriched Asparagus racemosus root extract comprising; (i) shatavarin I (ii) dehydroshatavarin I (iii) shatavarin IV (iv) shatavarin IX comprising blending said extracts with at least one component selected from pharmaceutically or nutraceutically or dietically acceptable excipients, carriers, and diluents.

In another embodiment, the present invention provides methods of obtaining at least one female reproductive and/or sexual health benefits selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation; wherein the method comprises supplementing the human subject in need thereof with an effective dose of a Asparagus racemosus composition comprising; (i) shatavarin I; (ii) dehydroshatavarin I; (iii) shatavarin IV; (iv) shatavarin IX and (v) optionally with at least one component selected from pharmaceutically or nutraceutically or dietically acceptable excipients, carriers, and diluents.

In yet another embodiment, the present disclosure provides use of the Asparagus racemosus composition comprising; (i) shatavarin I; (ii) dehydroshatavarin I; (iii) shatavarin IV; (iv) shatavarin IX and (v) optionally with at least one component selected from pharmaceutically or nutraceutically or dietically acceptable excipients, carriers, and diluents; for obtaining at least one female reproductive and sexual health benefit selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation.

### Methods of Use:

Estrogen receptor-α (ER-α): The cyclic secretion of estrogen and its interaction with relevant receptors, estrogen receptor-α and β (ER-α and ER-β), play a prominent role in ovarian function, regulation of endometrial proliferation and differentiation. Recently, the G-protein-coupled estrogen receptor (GPER) has been described as mediating the non-genomic signalling of estrogen. Changes in ER-α signalling pathways affect cellular activities, such as ovulation, cell cycle phase, cell proliferation, migration, and invasion. Further, chronic disturbance in female hormones affects ovarian function, which leads to the formation of cysts in the ovary and results in polycystic ovary syndrome (PCOS). Similarly, the endocrine and metabolic abnormalities related to PCOS can increase the risk of endometrial hyperplasia and cancer in women. Thus, impaired fertility in patients with PCOS may result not only from anovulation but also from endometrial dysfunction. Selective estrogen receptor modulators (SERMs) that are non-steroidal drugs have been widely used in clinical and therapeutic applications in the management of hormonal imbalance and PCOS in women. SERMs can act as both ER agonists and antagonists. However, sustained activation of ER-α by ER agonists has several side effects, including ER-α positive cancers. Alternatively, safer options, including phytoestrogens such as isoflavones, prenyl flavonoids, coumestans, and lignans, are increasing for the management of hormonal imbalances resulting in several women's health conditions, including PCOS.

The four compounds of Asparagus racemosus root Shatavarin I, dehydroshatavarin I, shatavarin IV, and shatavarin IX, and their combinations were evaluated for their ability to enhance the pER-α (estrogen receptor alpha phosphorylated at Serine 118 position) expression in a human breast tumor cell line, MCF-7, and the results are presented in Table 3. pER-α represents the active form of the hormone receptor. Unexpectedly, the combination of four markers showed better efficacy in increasing pER-α expression when compared to the corresponding individual markers.

TABLE 3
Normalized pER-α expression of
the four markers and their combination.

| | | | | % increase of |
|---|---|---|---|---|
| | | | pER-α | pER-α (from |
| | Product | Conc | expression in | vehicle |
| Marker name | code | (μg/mL) | arbitrary units | control) |
| Vehicle control | DMSO | 0.2% (v/v) | 0.28 | — |
| Shatavarin I | ARM-1 | 0.2 | 0.34 | 21.43 |
| Dehydro | ARM-2 | 0.2 | 0.45 | 60.71 |
| shatavarin I | | | | |
| Shatavarin IV | ARM-3 | 0.2 | 0.40 | 42.86 |
| shatavarin IX | ARM-4 | 0.2 | 0.38 | 35.71 |
| Combination of | ARM-1 + | 0.2 | 0.55 | 96.43 |
| four markers | ARM-2 + | | | |
| | ARM-3 + | | | |
| | ARM-4 | | | |
| | (1:1:1:1:1) | | | |

Note:
Higher the value indicates better efficacy

Thus, pER-α expression of shatavarin I, dehydroshatavarin I, shatavarin IV, and shatavarin IX each at 0.2 μg/mL concentrations showed 21.43%, 60.71%, 42.86% and 35.71% increases in pER-α expressions from the vehicle control; When compared with other markers, the novel marker dehydroshatavarin I showed better efficacy, which is a surprising result. The inventive combination of all four markers at the same concentration showed 93.46% increase in pER-α expression in comparison with individual markers, which is a surprising and unexpected result for ER-α activation.

Further, Asparagus racemosus root water extract (comp. example 1), 90% aqueous ethanol (comp. example 2), and inventive composition (example 1) are screened for its pER-α expression activity, and the results are presented in Table 4. Unexpectedly, the inventive composition showed better efficacy in pER-α expression when compared to the corresponding water extract and 90% aqueous ethanol extract.

TABLE 4
Normalized pER-α expression of Asparagus racemosus root
water extract, 90% aqueous ethanol extract, and inventive composition.

| | | | | % increase of |
|---|---|---|---|---|
| | | | pER-α | pER-α (from |
| Example | Product | Conc | expression | vehicle |
| ≠ | code | (μg/mL) | (arbitrary units) | control) |
| Vehicle control | DMSO | 0.2% | 0.26 | — |
| | | (v/v) | | |
| Comp ex 1 | AR-1 | 0.2 | 0.32 | 23.08 |
| (Water extract) | | | | |
| Comp ex 2 | AR-2 | 0.2 | 0.34 | 30.77 |
| (90% aq. | | | | |
| ethanol extract) | | | | |
| Example-13 | Comp-1 | 0.2 | 0.41 | 57.69 |
| (AR-3 + AR-4 | (4:1) | | | |

Note:
Higher the value indicates better efficacy

Thus, pER-α expression of water extract (AR-1) and 900% aqueous ethanol extract of Asparagus racemosus (AR-2) at each 0.2 g/mL concentrations showed 23.08% and 30.77% increase in pER-α expression, respectively, when compared to the vehicle control (Table 4). Whereas the inventive extract composition comprising enriched water extract of Asparagus racemosus obtained with 90% aq. ethanol and spent Asparagus racemosus raw material extract obtained with 90% aq. ethanol (Comp-1) at the same concentration showed a 57.69% increase in pER-α expression, which is a surprising and unexpected result for increased ER-α activation in the cells.

Prostaglandin E₂ (PGE₂): Prostaglandins (PGs) are lipid compounds called eicosanoids, which are derived from arachidonic acid released from lipid membranes following activation of phospholipase A2 enzyme. Cyclooxygenases-1 and 2 (COX-1 and COX-2) are the enzymes responsible for prostaglandin E₂ (PGE₂) production. Higher PGE₂ levels increase the sensitivity of peripheral nociceptive primary afferent neurons and central nociceptive neurons, which may be responsible for the development of uterine cramp resulting in menstrual pain (dysmenorrhea). The pharmacological therapy for dysmenorrhea is the usage of nonsteroidal anti-inflammatory drugs (NSAIDs), such as ibuprofen, which blocks PGE2 production by inhibiting COX enzymes. However, long-term usage of NASAIDs results in undesirable side effects. Hence, alternative strategies such as counter-pressure regio sacralis therapy, natural supplements are explored to manage dysmenorrhea.

Hence, Asparagus racemosus root water extract (AR-1), and inventive composition (Comp-1) are screened for its PGE₂ inhibition activity, and the results are presented in Table 5. Unexpectedly, the inventive composition showed better efficacy in inhibition of PGE₂ production when compared to the corresponding water extract.

TABLE 5
PGE2 inhibition by the Asparagus racemosus
water extract and inventive composition

| | | % reduction of | % Improvement |
|---|---|---|---|
| | Product | PGE₂ production at | from regular |
| Exam # | code | 10 μg/mL | extract (AR-1) |
| 1 | AR-1 | 15.23 | — |
| 2 | Comp-1 | 39.71 | 24.48 |

Thus, water extract of Asparagus racemosus (AR-1) at 10 μg/mL showed a 15.23% reduction in PGE₂ production; whereas, the inventive composition comprising enriched water extract of Asparagus racemosus obtained with 90% aq. ethanol and spent Asparagus racemosus raw material extract obtained with 90% aq. ethanol (comp-1) at the same treatment concentration showed 39.71 inhibition (Table 5), which is 24.48% higher activity than AR-1 and this is a surprising and unexpected result of PGE₂ inhibition.

Estrone: Perimenopause or menopause transition is conventionally classified into early and late phases. Understanding the biological mechanisms of perimenopause is a complicated process. The perimenopause-associated physiological changes are primarily due to the endocrine changes during perimenopause. A decline in the number of ovarian follicles results in decreased levels of inhibin B, anti-Mullerian hormone and ovarian estrogens [estrone (E1), 17β-estradiol (E2), estriol (E3)]. It is known that estrogens confer natural protection during the premenopausal phase. Menopausal women have very low circulating levels of estrogens. Estrone is one of the estrogens which converts into 17β-estradiol (E2) and helps in hormonal balance. Several pharmacological strategies and herbal supplements (containing phytoestrogens) that target perimenopause/menopause and associated metabolic disorders focused on hormonal balance, specifically, estrogens and progesterone (as demonstrated in Table-6 of example 7).

Evaluation of Asparagus racemosus enriched extract compositions supplementation on total follicular count of female Sprague Dawley rats: Ovarian follicles contain oocytes and surrounded by granulosa cells. Four different types of follicles are observed in ovaries based on their development stages: primordial, primary, secondary, and tertiary (antral). The number of primordial follicles, which is the true ovarian reserve, is determined in the fetus and declines throughout life. However, the primordial follicles cannot be visualized by ultrasound due to their small size (<0.05 mm diameter). The antral or Graafian follicles are more easily identifiable by ultrasound when they reach 2 mm in diameter. It is recommended that a follicular count needs to be performed in the early follicular phase of the menstrual cycle. However, considering the intracycle fluctuation of ovarian reserve, a follicular count may be performed at any point in the menstrual cycle. Antral follicles (AF) or Graafian follicles (GF) are considered as the best clinical markers for the assessment of functional ovarian reserve and indicator of fertility. This helps to predict the response to gonadotropin stimulation and the chance of pregnancy following in vitro fertilization (IVF). The number of GF decreases with age and has the potential to help predict the timing of natural menopause. The efficacy of the inventive extract composition (comp-1) was compared to the simple water extract (AR-1) in an in vivo experiment in female Sprague Dawley rats, as described in example 28. The data from the in vivo study unexpectedly showed better efficacy for the composition in increasing the total follicular count when compared to the water extract of Asparagus racemosus, suggesting that the enriched Asparagus racemosus extract composition tends to exhibit better efficacy (as demonstrated in Table 16 of example 28).

Hence, the inventive Asparagus racemosus root enriched extract composition containing Shatavarin I, dehydroshatavarin I, shatavarin IV, and shatavarin IX; obtained by the combination of enriched water extract of Asparagus racemosus obtained with 90% aq. ethanol and spent Asparagus racemosus raw material extract obtained with 90% aq. ethanol, surprisingly, showed a better efficacy in pER-α expression and PGE2 inhibition when compared to the regular water extract and 90% aqueous ethanol extract. Furthermore, surprisingly, the enriched Asparagus racemosus extract composition showed better efficacy in increasing the total follicular count in the experimental rats.

### Compositions

Several compositions were prepared comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%.

For example, various compositions of enriched water extract of Asparagus racemosus root obtained with 90% aq. ethanol (AR-3) and spent or residual Asparagus racemosus (Shatavari) root extract obtained with 90% aqueous ethanol (AR-4) were prepared, as shown in example 13. Similarly, various compositions of enriched water extract of Asparagus racemosus root obtained with polar organic solvents and spent or residual Asparagus racemosus (Shatavari) root obtained with polar organic solvent were prepared, as shown in examples 14-19.

Further, various compositions of enriched water extract of Asparagus racemosus root obtained through macroporous resin process by eluting with 90% aqueous ethanol (AR-11) and spent or residual Asparagus racemosus root extract obtained with 90% aqueous ethanol (AR-4) were prepared, as shown in example 20. Similarly, various compositions of enriched water extract of Asparagus racemosus root obtained through macroporous resins process by eluting with polar organic solvent and spent or residual Asparagus racemosus root extract obtained with other polar organic solvents were prepared, as shown in examples 21-22.

Further, various compositions of (i) water extract of Asparagus racemosus root (AR-1); (ii) enriched water extract of Asparagus racemosus root obtained through macroporous resin process by eluting with 90% aqueous ethanol (AR-11) and (iii) spent or residual Asparagus racemosus root 90% aqueous ethanol extract (AR-4) were prepared, as shown in example 23.

Further, these compositions (1 to 25) were evaluated for female health benefits selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, menstrual cramps; alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation.

pER-α expression activity: These compositions (1 to 25) were evaluated for Normalized pER-α expression activity in comparison with the corresponding individual ingredients (Example 26). The data from in vitro pER-α expression revealed that these compositions unexpectedly showed better efficacy when compared to their corresponding individual ingredients, suggesting that the compositions comprising; (i) shatavarin I (ii) dehydroshatavarin I (iii) shatavarin IV and (iv) shatavarin IX; obtained by combining (a) enriched water extract of Asparagus racemosus root obtained either through solvent extraction process or macroporous resin column chromatographic process and (b) spent or residual Asparagus racemosus (Shatavari) root polar organic solvent extract, tend to exhibit synergism.

For example, Asparagus racemosus root water extract enriched with 90% aq. ethanol (AR-3) at 0.16 μg/mL and 90% aq. ethanol (AR-4) extract of the spent raw material at 0.04 μg/mL showed 0.42 and 0.09 expression of pER-α, respectively. The composition-1 containing these two extracts at 4:1 ratio showed 0.72 expression of pER-α at 0.2 μg/mL concentration, which is significantly better than the additive effect (0.42+0.09=0.51) from these two ingredients, suggesting a synergistic effect between Asparagus racemosus root water extract enriched with 90% aq. ethanol (AR-3) and 90% aq. ethanol (AR-4) extract of the spent raw material. The compositions 2-5 obtained when combining these two ingredients at ratios 3:1, 2:1, 1:1, and 1:2, respectively, also showed synergism in pER-α expression compared to the expression shown by their corresponding individual ingredients as summarized in Table 8. Similarly, the other compositions (6-25) also showed synergism in pER-α expression compared to the expressions shown by their corresponding individual ingredients, as summarized in Tables 9-11.

Prostaglandin E₂ (PGE₂) inhibition activity: These compositions (1 to 25) were evaluated for PGE₂ inhibition activity in comparison with the corresponding individual ingredients (Example 27). The data from in vitro PGE₂ inhibition revealed that these compositions unexpectedly showed better inhibition when compared to their corresponding individual ingredients, suggesting that the compositions comprising; (i) shatavarin I (ii) dehydroshatavarin I (iii) shatavarin IV and (iv) shatavarin IX; obtained by combining (a) enriched water extract of Asparagus racemosus root produced either through solvent extraction process or macroporous resin column chromatographic process and (b) spent or residual Asparagus racemosus (Shatavari) root polar organic solvent extract, tend to exhibit synergism.

For example, enriched 90% aq. ethanol (AR-3) extract obtained from water extract of Asparagus racemosus root at 8.0 μg/mL concentration and 90% aq. ethanol (AR-4) extract of spent raw material at 2.0 μg/mL concentration showed 18.66% and 5.94% inhibition of PGE₂, respectively. The composition-1 containing these two extracts at 4:1 ratio showed 39.71% inhibition of PGE₂ at 10 μg/mL concentration, which is significantly better than the additive effect (18.66+5.94=24.60%) from these two ingredients, suggesting a synergistic effect between enriched 90% aq. ethanol (AR-3) extract obtained from water extract of Asparagus racemosus root and 90% aq. ethanol (AR-4) extract of spent raw material. The compositions 2-5 obtained when combining these two ingredients at ratios 3:1, 2:1, 1:1, and 1:2, respectively, also showed synergism in PGE₂ inhibition compared to the inhibition shown by their corresponding individual ingredients, as summarized in Table 12. Similarly, the other compositions (6-25) also showed synergism in PGE₂ inhibition compared to the inhibition shown by their corresponding individual ingredients, as summarized in Tables 13-15.

Formulation: The synergistic enriched compositions comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0% described; can be formulated using pharmaceutically or nutraceutically, or dietically acceptable excipients, carriers, and diluents; selected from monosaccharides, disaccharides, polysaccharides, dextrins, polyhydric alcohols or sugar alcohols, cellulose-based derivatives, silicates, metallic stearates, organic acids, fatty acid esters, calcium pantothenate, amino acids, proteins, organic metal salts, natural pigments, flavours, Class I & Class II preservatives and mixtures thereof.

Monosaccharides are selected from but not limited to glucose, fructose, galactose; Disaccharides are selected from but not limited to sucrose, maltose, lactose, lactulose, trehalose cellobiose, chitobiose; Polysaccharides are selected from but not limited to starch and modified starch such as sodium starch glycolate, pre-gelatinized starch, soluble starch, ultra-sperse A and other modified starches; Dextrins are selected from but not limited to yellow dextrin, white dextrin, maltodextrin, glucidex 12D, rice maltodextrin, Tapioca/Cassava maltodextrin; Polyhydric alcohols or sugar alcohols are selected from but not limited to sorbitol, mannitol, inositol, xylitol, isomalt; Cellulose based derivatives are selected from but not limited to microcrystalline cellulose, hydroxy propyl methyl cellulose, hydroxy ethyl cellulose; Silicates are selected from but not limited to neusilin, veegum, talc, colloidal silicon dioxide, syloids; Metallic stearates are selected from but not limited to calcium stearate, magnesium stearate, zinc stearate; Organic acids are selected from but not limited to citric acid, tartaric acid, malic acid, succinic acid, lactic acid, L-ascorbic acid; Natural gums are selected from but not limited to acacia, carrageenan, guar gum, xanthan gum, Gum Ghatti etc. Bio polymers/Bio polysaccharides such as sodium Alginate, calcium alginate, and alginic acid; Proteins are selected from but not limited to whey protein, whey isolate, casein, gelatine, pectin, and agar; inorganic metal salts are selected from but not limited to sodium chloride, calcium chloride, dicalcium phosphate, zinc sulphate, zinc chloride, natural sweeteners such as licorice, Stevia, Steviosides such as Rebaudioside A, Palm Sugars, and natural sweet proteins such as but not limited to Brazzein, Monellin, Curculin, Mabinlin, Miraculin, and Pentadin, etc.

For example, the comp-26 was prepared by combining AR-3 and AR-4 with Gum acacia and colloidal silicon dioxide. Similarly, other compositions (comp 27-29) also were prepared as shown in example 25.

Process: The process for the preparation of synergistic enriched Asparagus racemosus composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and (v) optionally with at least one component selected from pharmaceutically or nutraceutically or dietically acceptable excipients, carriers, and diluents; comprising the following steps of;
(a) extracting Asparagus racemosus root powder with water;
(b) evaporating the water extract to a minimum volume to obtain the concentrate;
(c) re-extracting the step (b) concentrate with a polar organic solvent;
(d) evaporating the polar organic solvent soluble extract to give an enriched extract;
(e) alternately passing the water extract of step (a) on adsorbent resin and then eluting the resin with aqueous ethanol or subjected to membrane filtration;
(f) evaporating the aqueous ethanol eluent to give an enriched extract;
(g) extracting the Asparagus racemosus root spent residue from step (a) with a polar organic solvent;
(h) evaporating the polar organic solvent extract in step (g) to give a second extract;
(i) combining the extracts of step (d) or step (f) and step (h) to obtain synergistic enriched Asparagus racemosus extract composition; and
(j) optionally blending the combined extract of step (i) with at least one component selected from pharmaceutically or nutraceutically or dietically acceptable excipients, carriers and diluents.

The polar organic solvent used in the process for the preparation of synergistic enriched Asparagus racemosus extract composition is selected from but not limited to ethanol, n-butanol, methanol, acetone, aqueous ethanol, aqueous methanol, aqueous acetone and mixtures thereof.

Evaluation of Asparagus racemosus synergistic enriched extract compositions (comp-26, comp-27, comp-28 and 90% aqueous ethanol extract (AR-2) supplementation in letrozole-induced PCOS in female Sprague Dawley rats: Oral glucose tolerance test (OGTT): OGTT measures circulatory glucose levels following glucose ingestion, and this test is widely used as an indicator of insulin resistance. PCOS is known to cause metabolic dysfunctions, including insulin resistance. Letrozole-induced rats show impaired glucose tolerance and increased blood glucose in circulation, which closely mimics the clinical features of glucose intolerance or insulin resistance in PCOS.

Area Under the Curve (AUC) was calculated using serum glucose concentration Vs. time (in min). The AUC value was increased in letrozole-induced (G2) rats when compared with the Vehicle control (G1) rats, indicating impaired glucose tolerance or increased insulin resistance. AUC values in AR-2 and comp 26, 27, and 28 supplemented rats showed 9.70%, 13.51%, 16.62%, and 20.05% improvements, respectively, from the G2 rats. These improvements indicate increased glucose tolerance in the Comp-26, Comp-27, and Comp-28 supplemented rats, and these effects are significantly better than the AR-2 group of rats (Table 17).

Serum levels of hormones: Letrozole is an aromatase inhibitor; it increases serum testosterone levels and reduces estrogen levels, resulting in hyperandrogenism or a condition of excess androgen or male hormones. Hyperandrogenism is a common feature and is prevalent in more than 80% of women suffering from PCOS.

Estradiol: 17β-Estradiol (E2) is a female sex hormone, produced in ovaries, has influence on the physical and psychological well-being of women. E2 plays a key role in the development of the female reproductive system and exerts its effects on target organs and cells via multiple estrogen receptors (ERs), ERα, ERβ, and the G-protein-coupled estrogen receptor (GPER, also known as GPR30) to maintain various stages of normal development in the human ovaries and uterus. E2 levels dramatically change at hormonal transitions, such as puberty, menopause, and postpartum. At these transitions' women are at increased risk for psychological and somatic distress. Women have higher amounts of E2 during their reproductive years, and gradually decreases after menopause. Balancing/improving the levels of E2 would be helpful for the management of conditions such as peri-menopause and polycystic ovary syndrome (PCOS).

Testosterone: In women, androgens, including testosterone, are produced by the ovaries and adrenal glands, as well as by conversion of less potent to more potent androgens in the periphery. Women with PCOS show hyperandrogenism and have significantly elevated levels of testosterone, free testosterone, androstenedione, dehydroepiandrosterone (DHEA), and DHEA sulfate (DHEAS) compared to ovulatory and non-hirsute women. The hyperandrogenism results in a wide spectrum of manifestations ranging from mild acne, increased terminal (coarse) hair growth in midline structures (face, neck, abdomen), to android changes in body habitus, with waist-to-hip ratio. The primary goal of all forms of therapy is to suppress insulin-facilitated, LH-driven androgen production, including testosterone. Therefore, balancing or improving the E2 and testosterone ratio would alleviate the clinical symptoms of ovarian dysfunction, including PCOS.

Serum samples from all experimental rats were analyzed for 17β-Estradiol and Testosterone. In Letrozole-induced PCOS rats (G2), the serum estradiol levels were decreased (32.97±13.89 μg/ml vs. 41.35±23.93 μg/ml in G1), and the testosterone levels were increased (3.18±2.16 μg/ml vs. 0.26±0.10 in G1). The comp 26, 27, and 28 supplemented rats showed 10.74%, 13.95%, and 19.20% improvements in serum 17β-Estradiol, and 43.08%, 60.38%, and 70.44% reductions in serum testosterone levels from the G2 rats, respectively. AR-2 increased 17β-Estradiol by 7.24% and reduced testosterone levels by 30.50% compared to the G2 rats. These data indicate that comp 26, 27, or 28 supplementation helps improve the hormonal balance in the experimental PCOS rats, and these improvements are significantly better than the AR-2 supplemented rats. The serum hormone analysis data are presented in Table 18.

Follicular cysts and total number of follicles: PCOS is a heterogeneous disorder that affects at least 7% of adult women. Formation of ovarian cysts is one of the symptoms of the PCOS along with increased levels of androgens and irregular or skipped periods. The word “polycystic” means “many cysts” referring to the formation of many small, fluid-filled sacs that grow along the outer edge of the ovaries. These sacs are follicles containing immature eggs that never mature to trigger ovulation. The lack of ovulation alters the levels of estrogen, progesterone, FSH, LH and increases the androgen levels. Balancing the hormone levels in conditions such as PCOS, peri-menopause, or menopause, along with improving insulin sensitivity, will improve ovulation and possibly reduce cyst formation.

In the experimental animals, the ovary sections were examined for follicular cysts and the total number of follicles, including the Graafian follicles. Letrozole significantly reduced the total number of follicles and the Graafian follicles and increased the cyst count in the ovaries of the experimental rats. The comp 26, 27, and 28 supplementations significantly increased the total number of follicles by 30.67%, 46.92% and 49.59%, and the number of the Graafian follicles by 28.29%, 38.05%, and 70.73% respectively, and reduced the number of cysts by 38.06%, 46.82%, and 50.66%, respectively, as compared to the Letrozole-induced PCOS rats. In contrast, AR-2 supplemented rats showed 19.56% and 16.59% increases in total follicles and the Graafian follicle counts, respectively, and a 27.38% decrease in the number of follicular cysts, as compared to the G2 rats. These improvements in the comp 26, 27, and 28 supplemented rats are significantly better than the AR-2 supplemented rats. The observations on follicular cysts and the total number of follicles, including the Graafian follicles, are summarized in Table 19.

Thus, the present observations suggest that comp 26, 27, 28, and AR-2 improve the glucose intolerance in the Letrozole-induced PCOS rats. The incidence of hyperglycemia due to glucose intolerance or insulin resistance is highly correlated and a prominent feature of PCOS in humans. The observations also suggest that AR-2 and comp 26, 27, and 28 significantly alleviated the clinical presentations of PCOS, such as the number of follicles and cysts in the experimental rats. Also, the test products (compositions and the regular extracts) are efficacious in improving the hormonal balance (estrogen vs. testosterone) and the Graafian follicular counts in the experimental animals. This is of note that a higher count of Graafian follicles or mature follicles is an established clinical marker of improved fertility. Importantly, comp 26, 27, and 28 are significantly superior to the regular extract in improving ovarian function to alleviate the PCOS condition (number of cysts, follicles), reduce hyperandrogenism, and improve hormonal balance and fertility.

Thus, a synergistic enriched Asparagus racemosus (Shatavari) root extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and (v) optionally at least one component selected from pharmaceutically or nutraceutically or dietically acceptable excipients, carriers, and diluents; showed female health benefits.

Therefore, in an important embodiment, the present invention provides synergistic enriched Asparagus racemosus root extract composition comprising; (i) shatavarin I (ii) dehydroshatavarin I (iii) shatavarin IV, and (iv) shatavarin IX; for female health benefits.

In one of the preferred embodiments, the invention provides hither to un reported a novel shatavari compound, i.e., Dehydroshatavarin compound I of the formula;

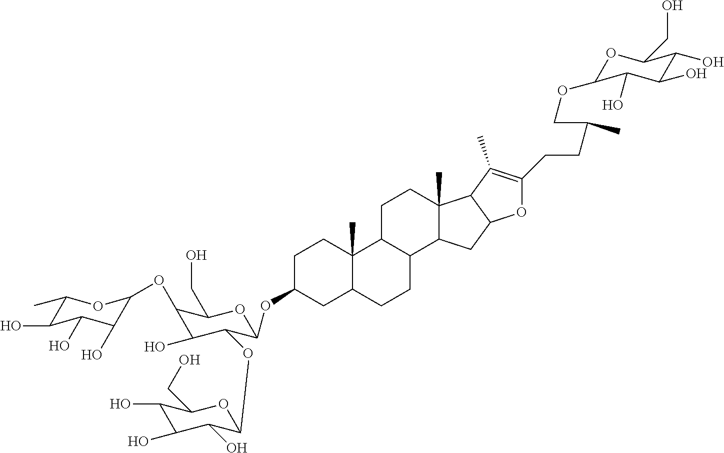

Characterized by ¹H NMR (d₆-DMSO): δ 5.16 (1H, d, J=4.4 Hz), 5.13 (1H, d, J=4.0 Hz), 4.90 (1H, d, J=4.8 Hz), 4.81-4.85 (4H, m), 4.72 (1H, s), 4.62-4.68 (4H, m), 4.59 (1H, d, J=5.2 Hz), 4.47 (1H, d, J=8.0 Hz), 4.41 (1H, d, J=5.6 Hz), 4.37 (1H, t, J=6.4 Hz), 4.33 (1H, d, J=7.6 Hz), 4.27-4.32 (1H, m), 4.16 (1H, t, J=5.6 Hz), 4.08 (1H, d, J=8.0 Hz), 3.64-3.67 (3H, m), 3.61 (1H, brs), 3.57-3.58 (1H, m), 3.37-3.51 (7H, m), 3.03-3.19 (9H, m), 2.92-3.01 (2H, m), 2.01-2.13 (3H, m), 1.63-1.84 (5H, m), 1.55 (3H, s), 1.36-1.52 (9H, m), 1.15-1.28 (5H, m), 1.10 (3H, d, J=6.4 Hz), 1.06-1.09 (2H, m), 0.95-1.05 (2H, m), 0.90 (3H, s), 0.89 (3H, d, J=6.4 Hz), 0.62 (3H, s). Mass (Q-tof, negative ion mode): m/z 1047.5400 (M−H)⁻

In one preferred embodiment, the present invention provides synergistic enriched Asparagus racemosus extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; for female health benefits.

In one preferred embodiment, the present invention provides synergistic enriched Asparagus racemosus extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; wherein the said composition is produced by combining (a) enriched water extract of Asparagus racemosus (Shatavari) root obtained either through polar organic solvent extraction or purification on macroporous resins and (b) spent or residual Asparagus racemosus (Shatavari) root polar organic solvent extract.

In one preferred embodiment, the present invention provides synergistic enriched Asparagus racemosus root extract composition comprising; (i) shatavarin I in the range of 0.5-15%; (ii) dehydroshatavarin I in the range of 0.1-25%; (iii) shatavarin IV in the range of 2-25%; and (iv) shatavarin IX in the range of 0.001-1.0%; and (v) further containing optionally at least one additional component selected from the group consisting of biological agent(s), and Nootropic agent(s); pharmaceutically acceptable active ingredients, vitamins, minerals; pharmaceutically or nutraceutically or dietically acceptable, excipients, carriers or diluents.

In another embodiment, the present invention provides synergistic enriched Asparagus racemosus extract composition comprising, (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and (v) with at least one component selected from pharmaceutically or nutraceutically or dietically acceptable excipients, carriers, and diluents; wherein the Asparagus racemosus root extract composition varies in the range of 90%-10% by weight and excipients in the composition varies in the range of 10%-90% by weight.

In another embodiment, the present invention provides synergistic enriched Asparagus racemosus extract composition comprising, (i) shatavarin I; (ii) dehydroshatavarin I; (iii) shatavarin IV, and (iv) shatavarin IX; and (v) optionally containing at least one additional ingredient selected from shatavarin II, shatavarin III, shatavarin V, shatavarin VI, shatavarin VII, shatavarin VIII, shatavarin IX, asparacoside, asparanin and immunoside or mixtures thereof.

In another embodiment, the present invention provides synergistic enriched Asparagus racemosus extract composition comprising; (i) shatavarin I; (ii) dehydroshatavarin I; (iii) shatavarin IV and (iv) shatavarin IX; and (v) optionally containing at least one extract or fraction selected from Nigella sativa, Cinnamomum zeylanicum, Foeniculum vulgare, Trigonella foenum-graecum, Glycyrrhiza glabra and Linum usitatissimum or atleast one phytochemical selected from Inositols such as myo-inositol or D-chiro-inositol or mixtures thereof.

In yet another embodiment, the present invention provides process for the preparation of synergistic enriched Asparagus racemosus extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and (v) optionally with at least one component selected from pharmaceutically or nutraceutically or dietically acceptable excipients, carriers, and diluents; which comprises the steps of;
(a) extracting Asparagus racemosus root powder with water;
(b) evaporating the water extract to minimum volume to obtain the concentrate;
(c) re-extracting the step (b) concentrate with a polar organic solvent;
(d) evaporating the polar organic solvent soluble extract of step (c) to give an enriched extract;
(e) alternately passing the water extract of step (a) on adsorbent resin and then eluting with aqueous ethanol;
(f) evaporating the aqueous ethanol eluent of step (e) to give an enriched extract;
(g) extracting the Asparagus racemosus root spent residue from step (a) with a polar organic solvent;
(h) evaporating the polar organic solvent extract in step (g) to give a second extract;
(i) combining the enriched extracts of step (d) or step (f) and step (h) to obtain synergistic enriched Asparagus racemosus root extract composition; and
(j) optionally blending the synergistic enriched Asparagus racemosus root extract composition of step (i) with at least one component selected from pharmaceutically or nutraceutically or dietically acceptable excipients, carriers and diluents.

The polar organic solvent used in the process for the preparation of synergistic enriched Asparagus racemosus extract composition is selected from but not limited to ethanol, n-butanol, methanol, acetone, aqueous ethanol, aqueous methanol, aqueous n-butanol, aqueous acetone and mixtures thereof.

In yet another embodiment, the present invention provides synergistic enriched Asparagus racemosus extract composition comprising; (i) shatavarin I (ii) dehydroshatavarin I (iii) shatavarin IV and (iv) shatavarin IX; for female health benefits; wherein the said composition is prepared by a process which comprises the steps of, (a) enriching water extract of Asparagus racemosus (Shatavari) root by re-extracting with a polar organic solvent or by loading water extract on macroporous resin and eluting with an aqueous alcohol to obtain enriched extract; (b) extracting spent or residual Asparagus racemosus (Shatavari) root with a polar organic solvent to obtain second extract; (c) combining the extracts obtained from step (a) and step (b); and (d) optionally blending with at least one component selected from pharmaceutically or nutraceutically or dietically acceptable excipients, carriers and diluents.

In yet another embodiment, water extract of Asparagus racemosus (Shatavari) root is also enriched through membrane filtration.

In yet another embodiment, the present invention provides synergistic enriched Asparagus racemosus extract composition comprising; (i) shatavarin I in the range of 0.5-15%; dehydroshatavarin I in the range of 0.1-25%; shatavarin IV in the range of 2-25% and shatavarin IX in the range of 0.001-1.0%; for female health benefits; wherein the said composition is prepared by a process which comprises the steps of, (a) extracting Asparagus racemosus root with water, (b) enriching the water extract of Asparagus racemosus (Shatavari) root by re-extracting with a polar organic solvent or by loading water extract on macroporous resin and eluting with an aqueous alcohol to obtain enriched extract; (c) extracting spent or residual Asparagus racemosus (Shatavari) root with polar organic solvent to obtain second extract; (d) combining the extracts obtained from step (b) and step (c); and (e) optionally blending with at least one component selected from pharmaceutically or nutraceutically or dietically acceptable excipients, carriers and diluents.

In another important embodiment, the present invention provides methods of obtaining at least one female reproductive and/or sexual health benefits selected from promoting or regulating the production of reproductive hormones to alleviate the hormonal deficiency and/or hormonal imbalance, reducing the symptoms of perimenopause, premenstrual syndromes (PMS), menopausal disorders including discomforts related to the menstrual cycle, alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation; wherein the method comprises supplementing the human subject in need thereof with an effective dose of a synergistic enriched Asparagus racemosus composition comprising; (i) shatavarin I in the range of 0.5-15%; dehydroshatavarin I in the range of 0.1-25%; shatavarin IV in the range of 2-25% and shatavarin IX in the range of 0.001-1.0%; and (v) optionally with at least one component selected from pharmaceutically or nutraceutically or dietically acceptable excipients, carriers, and diluents.

In yet another embodiment, the present disclosure provides use of the synergistic enriched Asparagus racemosus composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and (v) optionally with at least one component selected from pharmaceutically or nutraceutically or dietically acceptable excipients, carriers, and diluents; for obtaining at least one female reproductive and sexual health benefit selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation.

In another embodiment, the compositions as disclosed above; wherein the phytochemical reference marker compound or pharmacologically active marker or group of markers; may be estimated by several analytical techniques known in the art such as but not limited to HPLC, gravimetry, UV, GC, LC-MS and ICP-mass. In another embodiment, the synergistic enriched Asparagus racemosus compositions as disclosed above, wherein the composition is formulated into a dosage form selected from dry powder form, liquid form, beverage, food product, dietary supplement, or any suitable form such as a tablet, a capsule, a soft chewable tablet, gummies or gummy bar.

In another embodiment, the synergistic enriched Asparagus racemosus compositions as disclosed above, wherein the composition is formulated into a nutritional/dietary supplement that can be contemplated/made into the dosage form of healthy foods or food for specified health uses, such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

In another embodiment, the synergistic enriched Asparagus racemosus compositions as disclosed above, wherein the composition is formulated into a controlled-release tablet, using controlled-release polymer-based coatings by techniques including nanotechnology, microencapsulation, colloidal carrier systems, and other drug delivery systems for obtaining the desired therapeutic benefit.

### EXAMPLES

### Comparative Example 1 (AR-1): Asparagus racemosus Root Water Extract

Asparagus racemosus root (100 g) was pulverized and extracted with water (800 mL) at ambient temperature under maceration for 4 h. The water extract was filtered, and the process was repeated twice with water (2×600 mL) under similar extraction conditions for 4 h. The combined water extract was evaporated under reduced pressure to give the product as brown colour solid (AR-1, 45 g).

### Comparative Example 2 (AR-2): Asparagus racemosus Root 90% Aq. Ethanol Extract

Asparagus racemosus (100 g) was pulverized and extracted with 90% aq. ethanol (800 mL) at 80 °C for 4 h. The contents were cooled to RT and filtered. The extraction process was repeated with 90% aq. ethanol (800 mL) under similar extraction conditions. The combined 90% aq. ethanol extract was evaporated under reduced pressure to give the product as a brown color solid (AR-2, 42.0 g).

### Example 1: Enrichment of Water Extract with 90% Aq. Ethanol and Extraction of Spent with 90% Aq. Ethanol

AR-3: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h. The combined water extract was evaporated under reduced pressure to give the water extract. To this water extract was added 90% aq. ethanol (250 mL) and stirred at higher temperatures for 2 h. Separated the 90% aq. ethanol layer and was evaporated under reduced pressure to give the product as a brown color solid (23 g, AR-3).

AR-4: The spent raw material after water extraction was extracted with 90% aq. ethanol (600 mL) at higher temperatures for 2 h, and the mixture was cooled to rt and filtered. The extraction process was repeated twice with 90% aq. ethanol (2×600 mL) under similar extraction conditions. The combined 90% aq. ethanol solutions were evaporated under reduced pressure to give the product as a brown color solid (5.17 g, AR-4)

The solids from the above two extractions (AR-3 and AR-4) were combined and pulverized to obtain the enriched Asparagus racemosus root extract as a homogenous brown color solid. The inventive enriched Asparagus racemosus root extract was subjected to column chromatography over normal silica gel using solvents of increasing polarity. The fractions were further subjected to preparative HPLC using a reversed phase C18 silica column to obtain pure compounds. The structures of the pure compounds were established by analyzing the ¹HNMR, ¹³C NMR, and mass spectrometry data. Three of the pure compounds were identified as shatavarin I, shatavarin IV, and shatavarin IX by comparing the spectral data with those reported in the literature. The fourth compound was identified as dehydroshatavarin I, and surprisingly, this is not reported in the literature and it is a new compound.

Shatavarin I: ¹H NMR (d₆-DMSO): δ 5.17 (1H, d, J=4.4 Hz), 5.14 (1H, d, J=2.4 Hz), 5.00 (1H, s), 4.82-4.90 (5H, brs), 4.72 (1H, s), 4.59-4.63 (3H, m), 4.47 (1H, d, J=7.6 Hz), 4.38-4.43 (3H, m), 4.33 (1H, d, J=7.6 Hz), 4.16 (1H, t, J=5.6 Hz), 4.09 (1H, d, J=7.6 Hz), 3.92 (2H, brs), 3.88 (1H, dd, J=6.0, 9.6 Hz), 3.65-3.69 (3H, m), 3.57-3.61 (2H, m), 3.42-3.51 (8H, m), 3.16-3.19 (3H, m), 3.11-3.14 (2H, m), 3.05-3.10 (3H, m), 2.93-3.00 (2H, m), 1.79-1.93 (3H, m), 1.74 (2H, brs), 1.58-1.69 (5H, m), 1.35-1.55 (11H, m), 1.16-1.24 (2H, m), 1.06-1.15 (2H, m), 1.01 (3H, d, J=6.0 Hz), 0.95-0.98 (1H, m), 0.90-0.91 (3H, m), 0.90 (3H, s), 0.88 (3H, d, J=6.4 Hz), 0.80 (1H, s), 0.72 (3H, s). Mass (Q-tof, negative ion mode): m/z 1065.5480 (M−H)⁻.

Dehydroshatavarin I: ¹H NMR (d₆-DMSO): δ 5.16 (1H, d, J=4.4 Hz), 5.13 (1H, d, J=4.0 Hz), 4.90 (1H, d, J=4.8 Hz), 4.81-4.85 (4H, m), 4.72 (1H, s), 4.62-4.68 (4H, m), 4.59 (1H, d, J=5.2 Hz), 4.47 (1H, d, J=8.0 Hz), 4.41 (1H, d, J=5.6 Hz), 4.37 (1H, t, J=6.4 Hz), 4.33 (1H, d, J=7.6 Hz), 4.27-4.32 (1H, m), 4.16 (1H, t, J=5.6 Hz), 4.08 (1H, d, J=8.0 Hz), 3.64-3.67 (3H, m), 3.61 (1H, brs), 3.57-3.58 (1H, m), 3.37-3.51 (7H, m), 3.03-3.19 (9H, m), 2.92-3.01 (2H, m), 2.01-2.13 (3H, m), 1.63-1.84 (5H, m), 1.55 (3H, s), 1.36-1.52 (9H, m), 1.15-1.28 (5H, m), 1.10 (3H, d, J=6.4 Hz), 1.06-1.09 (2H, m), 0.95-1.05 (2H, m), 0.90 (3H, s), 0.89 (3H, d, J=6.4 Hz), 0.62 (3H, s). Mass (Q-tof, negative ion mode): m/z 1047.5400 (M−H)⁻.

Shatavarin LX: ¹H NMR (d₆-DMSO): δ 5.49 (1H, brs), 5.16 (2H, brs), 5.00 (1H, s), 4.94-4.96 (1H, m), 4.82 (4H, brs), 4.59-4.63 (1H, m), 4.38-4.51 (4H, m), 4.29 (1H, d, J=7.2 Hz), 4.09 (1H, d, J=8.0 Hz), 3.92 (2H, brs), 3.64-3.69 (5H, m), 3.42-3.51 (3H, m), 2.93-3.20 (8H, m), 1.80-1.93 (3H, m), 1.72 (2H, brs), 1.58-1.69 (5H, m), 1.35-1.55 (10H, m), 1.09-1.24 (7H, m), 0.90 (3H, s), 0.85-0.90 (6H, m), 0.72 (3H, s). Mass (Q-tof, positive ion mode): m/z 903.1861 (M+H)⁺.

Shatavarin IV: ¹H NMR (d₆-DMSO): δ 5.11 (1H, d, J=3.6 Hz), 5.07 (1H, d, J=3.6 Hz), 4.82 (1H, d, J=4.0 Hz), 4.48 (1H, d, J=4.0 Hz), 4.74 (1H, s), 4.53-4.56 (3H, m), 4.48 (1H, d, J=7.6 Hz), 4.32 (3H, brs), 4.09 (1H, brs), 3.79-3.93 (3H, m), 3.59-3.69 (3H, m), 3.43-3.51 (6H, m), 3.36 (2H, t, J=8.4 Hz), 3.21 (2H, brs), 3.09-3.13 (2H, m), 3.00 (1H, brs), 1.91 (2H, brs), 1.75-1.84 (5H, m), 1.67-1.69 (3H, m), 1.28-1.56 (12H, m), 1.16 (3H, d, J=6.4 Hz), 1.12 (3H, d, J=6.4 Hz), 1.01-1.10 (2H, m), 1.02 (3H, d, J=6.8 Hz), 0.94 (3H, d, J=6.8 Hz), 0.91 (3H, s), 0.72 (3H, s). LC-MS (positive ion mode): m/z 909 (M+Na)⁺.

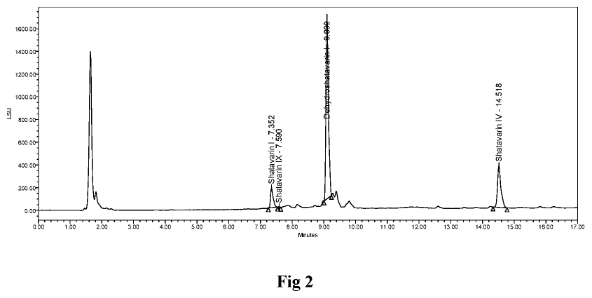

The pure compounds were then utilized as reference markers to standardize the extract. The pure compounds of Asparagus racemosus were evaluated using analytical HPLC method and their retention times (Rt) are summarized below in Table 6 below. The representative HPLC chromatogram of Asparagus racemosus extract composition is depicted in FIG. 2.

TABLE 6
Retention times of four markers

| S. No. | Compound name | Rt in min |
|---|---|---|
| 1 | Shatavarin I | 7.354 |
| 2 | Dehydroshatavarin I | 9.099 |
| 3 | Shatavarin IV | 14.518 |
| 4 | Shatavarin IX | 7.590 |

### Example 2: Enrichment of Water Extract with 90% Aq. Butanol and Extraction of Spent with 90% Aq. Ethanol

AR-5: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h. The combined water extract was evaporated under reduced pressure to 60 g of weight. To this residue was added 90% aq. n-butanol (250 mL) and stirred at 80 °C for 2 h. Separated the 90% aq. n-butanol layer and evaporated under reduced pressure to give the product as brown color solid (9 g, AR-5).

AR-4: The spent raw material after water extraction was extracted as described in example 1 to give the product as brown color solid (5.17 g, AR-4).

### Example 3: Enrichment of Water Extract with n-Butanol and Extraction of Spent with 90% Aq. Ethanol

AR-6: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h each. The combined water extract was evaporated under reduced pressure to 200 mL of volume. To this solution was added n-butanol (200 mL) and stirred at ambient temperature for 30 min. Separated the n-butanol layer and the process was repeated twice with n-butanol (2×200 mL). Combined n-butanol layer was evaporated under reduced pressure to give the product as brown color solid (11 g, AR-6).

AR-4: The spent raw material after water extraction was extracted as described in example 1 to give the product as brown colour solid (5.17 g, AR-4).

### Example 4: Enrichment of Water Extract with Methanol and Extraction of Spent with 90% Aq. Ethanol

AR-7: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h. The combined water extract was evaporated under reduced pressure to 60 g of weight. To this residue was added methanol (250 mL) and stirred at higher temperatures for 2 h. Separated the methanol layer and was evaporated under reduced pressure to give the product as brown color solid (34 g, AR-7).

AR-4: The spent raw material after water extraction was extracted as described in example 1 to give the product as brown colour solid (5.17 g, AR-4).

### Example 5: Enrichment of Water Extract with 80% Aq. Acetone and Extraction of Spent with 90% Aq. Ethanol

AR-8: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h. The combined water extract was evaporated under reduced pressure to 60 g of weight. To this residue was added 80% aq. acetone (250 mL) and stirred at higher temperatures for 2 h. Separated the 80% aq. acetone layer and evaporated under reduced pressure to give the product as a brown color solid (8 g, AR-8).

AR-4: The spent raw material after water extraction was extracted as described in example 1 to give the product as brown color solid (5.17 g, AR-4).

### Example 6: Enrichment of Water Extract with Methanol and Extraction of Spent with 90% Aq. Methanol

AR-7: Asparagus racemosus root (100 g) was pulverized, extracted with water and enriched with methanol by using the procedure as described in example 4 to give the product as brown color solid (34 g, AR-7).

AR-9: The spent raw material after water extraction was extracted with 90% aq. methanol (600 mL) at higher temperatures for 2 h. Cool to rt, and filter. The extraction process was repeated twice with 90% aq. methanol (2×400 mL) under similar extraction conditions. Combined 90% aq. methanol extract was evaporated under reduced pressure to give the product as a brown color solid (6.5 g, AR-9)

### Example 7: Enrichment of Water Extract with 80% Aq. Acetone and Extraction of Spent with 80% Aq. Acetone

AR-8: Asparagus racemosus root (100 g) was pulverized, extracted with water, and enriched with 80% aq. acetone by using the procedure as described in example 5 to give the product as brown color solid (8 g, AR-8).

AR-10: The spent raw material after water extraction was extracted with 80% aq. acetone (600 mL) at higher temperatures for 2 h. The contents were cooled to rt, and filtered. The extraction process was repeated twice with 80% aq. acetone (2×400 mL) under similar extraction conditions. The combined 80% aq. acetone extract was evaporated under reduced pressure to give the product as brown color solid (4.1 g, AR-10).

### Example 8: Enrichment of Water Extract with Macroporous Resin and Extraction of Spent with 90% Aq. Ethanol

AR-11: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h each. The combined water extract was loaded on PA-800 macroporous resin (125 mL, loading time 1 BV/h). After completion of loading, the column was eluted with water (250 mL, 2 BV/h). Following this, the column was further eluted with 90% aq. ethanol (750 mL, 2 BV/h), and the 90% aq. Ethanol eluents was evaporated under reduced pressure to give the product as brown color solid (5.0 g, AR-11).

AR-4: The spent raw material after water extraction was extracted with 90% aq. ethanol as described in example 1 to give the product as brown color solid (5.17 g, AR-4).

The solids from the above two extractions (AR-11 and AR-4) were combined and pulverized to obtain the enriched Asparagus racemosus root extract as a homogenous brown color solid.

### Example 9: Enrichment of Water Extract with Macroporous Resin and Extraction of Spent with 90% Aq. Methanol

AR-11: Asparagus racemosus root (100 g) was pulverized, extracted with water, and enriched with macroporous resin by using the procedure as described in example 8 to give the product as brown color solid (5.0 g, AR-11).

AR-9: The spent raw material after water extraction was extracted with 90% aq. methanol using the procedure as described in example 6 to give the product as brown color solid (6.5 g, AR-9)

### Example 10: Enrichment of Water Extract with Macroporous Resin and Extraction of Spent with 80% Aq. Acetone

AR-11: Asparagus racemosus root (100 g) was pulverized, extracted with water, and enriched with macroporous resin by using the procedure as described in example 8 to give the product as brown color solid (5.0 g, AR-11).

AR-10: The spent raw material after water extraction was extracted with 80% aq. acetone using the procedure as described in example 7 to give the product as brown color solid (4.1 g, AR-10)

### Example 11: Water Extraction of Asparagus racemosus Root, Enrichment of Water Extract with Microporous Resin and Extraction of Spent with 90% Aq. Ethanol

AR-1: Asparagus racemosus root (40 kg) was pulverized and extracted with water (320 L) at ambient temperature under maceration for 16 h. The water extract was filtered, and the process was repeated twice with water (2×240 L) under similar extraction conditions for 4 h. The combined water extract was divided into two parts. Part-1 of the solution was evaporated under reduced pressure to give the product as brown colour solid (8 kg, AR-1).

AR-11: part-2 of the solution was loaded on PA-800 macroporous resin (25 L, loading time 1 BV/h). After completion of loading, the column was eluted with water (50 L, 2 BV/h). Following this, the column was further eluted with 90% aq. ethanol (150 L, 2 BV/h), and the 90% aq. ethanol eluents was evaporated under reduced pressure to give the product as brown color solid (1.1 kg, AR-11).

AR-4: The spent raw material after water extraction was extracted with 90% aq. ethanol (240 L) at higher temperatures for 2 h. Cool to rt, and filter. The extraction process was repeated twice with 90% aq. ethanol (2×160 L) under similar extraction conditions. The combined 90% aq. Ethanol extract was evaporated under reduced pressure to give the product as brown color solid (2.2 kg, AR-4).

### Example 12: Enrichment of Water Extract Using Membrane Filtration and Extraction of Spent with 90% Aq. Ethanol

AR-12: Asparagus racemosus root (100 g) was pulverized and extracted with water (800 mL) at ambient temperature under maceration for 16 h. The water extract was filtered, and the process was repeated twice with water (2×600 mL) under similar extraction conditions for 4 h. The combined water extract was filtered through 0.5-micron filters and passed through ultra-filtration (10,000 Da) membrane. The ultra-filtration permeate was collected and evaporated under reduced pressure to give the product as brown color solid (20.16 g, AR-12) AR-4: The spent R/M after water extraction was extracted with 90% aq. ethanol (600 mL) at 80 °C for 12 h under continuous extraction process. The aq. ethanol extract after continuous extraction was evaporated under reduced pressure to give the product as brown color solid (4.13 g, AR-4)

### Example 13: Preparation of Various Compositions of Asparagus racemosus Root Containing Water Extract Enriched with 90% Aq. Ethanol (AR-3) and Spent 90% Aq. Ethanol Extract (AR-4) in the Following Ratios

Comp-1 (C-1): C-1 was prepared by combining AR-3 and AR-4 in the ratio of 4:1;
Comp-2 (C-2): C-2 was prepared by combining AR-3 and AR-4 in the ratio of 3:1;
Comp-3 (C-3): C-3 was prepared by combining AR-3 and AR-4 in the ratio of 2:1;
Comp-4 (C-4): C-4 was prepared by combining AR-3 and AR-4 in the ratio of 1:1;
Comp-5 (C-5): C-5 was prepared by combining AR-3 and AR-4 in the ratio of 1:2.

### Example 14: Preparation of Various Compositions of Asparagus racemosus Root Containing Water Extract Enriched with 90% Aq. Butanol (AR-5) and Spent 90% Aq. Ethanol Extract (AR-4) in the Following Ratios

Comp-6 (C-6): C-6 was prepared by combining AR-5 and AR-4 in the ratio of 2:1;
Comp-7 (C-7): C-7 was prepared by combining AR-5 and AR-4 in the ratio of 1:1;
Comp-8 (C-8): C-8 was prepared by combining AR-5 and AR-4 in the ratio of 1:2.

### Example 15: Preparation of Various Compositions of Asparagus racemosus Root Containing Water Extract Enriched with n-Butanol (AR-6) and Spent 90% Aq. Ethanol Extract (AR-4) in the Following Ratios

Comp-9 (C-9): C-9 was prepared by combining AR-6 and AR-4 in the ratio of 1:1.

### Example 16: Preparation of Various Compositions of Asparagus racemosus Root Containing Water Extract Enriched with Methanol (AR-7) and Spent 90% Aq. Ethanol Extract (AR-4) in the Following Ratios

Comp-10 (C-10): C-10 was prepared by combining AR-7 and AR-4 in the ratio of 1:1.

### Example 17: Preparation of Various Compositions of Asparagus racemosus Root Containing Water Extract Enriched with 80% Aq. Acetone (AR-8) and Spent 90% Aq. Ethanol Extract (AR-4) in the Following Ratios

Comp-11 (C-11): C-11 was prepared by combining AR-8 and AR-4 in the ratio of 1:1.

### Example 18: Preparation of Various Compositions of Asparagus racemosus Root Containing Water Extract Enriched with Methanol (AR-7) and Spent 90% Aq. Methanol Extract (AR-9) in the Following Ratios

Comp-12 (C-12): C-12 was prepared by combining AR-7 and AR-9 in the ratio of 1:1.

### Example 19: Preparation of Various Compositions of Asparagus racemosus Root Containing Water Extract Enriched with 80% Aq. Acetone (AR-8) and Spent 80% Aq. Acetone Extract (AR-10) in the Following Ratios

Comp-13 (C-13): C-13 was prepared by combining AR-8 and AR-10 in the ratio of 1:1.

### Example 20: Preparation of Various Compositions of Asparagus racemosus Root Containing Water Extract Enriched with Macroporous Resin (AR-11) and Spent 90% Aq. Ethanol Extract (AR-4) in the Following Ratios

Comp-14 (C-14): C-14 was prepared by combining AR-11 and AR-4 in the ratio of 2:1;
Comp-15 (C-15): C-15 was prepared by combining AR-11 and AR-4 in the ratio of 1:1;
Comp-16 (C-16): C-16 was prepared by combining AR-11 and AR-4 in the ratio of 1:2;

### Example 21: Preparation of Various Compositions of Asparagus racemosus Root Containing Water Extract Enriched with Macroporous Resin (AR-11) and Spent 90% Aq. Methanol Extract (AR-9) in the Following Ratios

Comp-17 (C-17): C-17 was prepared by combining AR-11 and AR-9 in the ratio of 2:1;
Comp-18 (C-18): C-18 was prepared by combining AR-11 and AR-9 in the ratio of 1:1;
Comp-19 (C-19): C-19 was prepared by combining AR-11 and AR-9 in the ratio of 1:2.

### Example 22: Preparation of Various Compositions of Asparagus racemosus Root Containing Water Extract Enriched with Macroporous Resin (AR-11) and Spent 80% Aq. Acetone Extract (AR-10) in the Following Ratios

Comp-20 (C-20): C-20 was prepared by combining AR-11 and AR-10 in the ratio of 2:1;
Comp-21 (C-21): C-21 was prepared by combining AR-11 and AR-10 in the ratio of 1:1;
Comp-22 (C-22): C-22 was prepared by combining AR-11 and AR-10 in the ratio of 1:2.

### Example 23: Preparation of Various Compositions of Asparagus racemosus Root Containing Water Extract (AR-1), Water Extract Enriched with Macroporous Resin (AR-11), and Spent 90% Aq. Ethanol Extract (AR-10) in the Following Ratios

Comp-23 (C-23): C-23 was prepared by combining AR-1, AR-11, and AR-4 in the ratio of 8:1:2
Comp-24 (C-24): C-24 was prepared by combining AR-1, AR-11, and AR-4 in the ratio of 1:1:1
Comp-25 (C-25): C-25 was prepared by combining AR-1, AR-11, and AR-4 in the ratio of 1:3:3.

### Example 24: Standardization of Asparagus racemosus Root Extracts

The various compositions of Asparagus racemosus root extracts were standardized to Shatavarin I, Dehydroshatavarin I, shatavarin IV, and Shatavarin IX by analytical HPLC, and the results are summarized in Table 7.

TABLE 7
Estimation of four shatavarins in compositions
(1-24) by HPLC method of analysis

| | Shatavarin | Dehydro | Shatavarin | Shatavarin | |
|---|---|---|---|---|---|
| Product | I | shatavarin I | IV | IX | Total |
| C-1 | 0.82% | 3.84% | 4.23% | 0.18% | 9.07% |
| C-2 | 0.81% | 3.74% | 5.14% | 0.18% | 9.87% |
| C-3 | 0.79% | 3.57% | 6.65% | 0.17% | 11.18% |
| C-4 | 0.76% | 3.25% | 9.68% | 0.14% | 13.83% |
| C-5 | 0.72% | 2.92% | 12.71% | 0.12% | 16.47% |
| C-6 | 5.44% | 5.84% | 7.51% | 0.53% | 19.31% |
| C-7 | 4.22% | 4.94% | 10.30% | 0.41% | 19.87% |
| C-8 | 3.02% | 4.05% | 13.03% | 0.30% | 20.40% |
| C-9 | 5.17% | 2.16% | 10.23% | 0.53% | 18.10% |
| C-10 | 1.64% | 1.88% | 9.67% | 0.10% | 13.29% |
| C-11 | 1.39% | 4.87% | 11.16% | 0.07% | 17.50% |
| C-12 | 2.74% | 1.12% | 5.71% | 0.16% | 9.73% |
| C-13 | 2.80% | 4.04% | 7.40% | 0.09% | 14.33% |
| C-14 | 6.46% | 12.76% | 11.22% | 0.03% | 30.47% |
| C-15 | 5.00% | 10.13% | 13.10% | 0.05% | 28.28% |
| C-16 | 3.55% | 7.51% | 14.99% | 0.06% | 26.11% |
| C-17 | 7.19% | 12.25% | 8.58% | 0.07% | 28.09% |
| C-18 | 6.10% | 9.37% | 9.14% | 0.09% | 24.72% |
| C-19 | 5.02% | 6.49% | 9.71% | 0.12% | 21.35% |
| C-20 | 7.39% | 12.20% | 8.71% | 0.04% | 28.35% |
| C-21 | 6.41% | 9.30% | 9.33% | 0.04% | 25.10% |
| C-22 | 5.42% | 6.40% | 9.96% | 0.07% | 21.85% |
| C-23 | 0.96% | 2.81% | 4.19% | 0.04% | 8.00% |
| C-24 | 3.45% | 7.26% | 8.99% | 0.04% | 19.74% |
| C-25 | 4.34% | 8.90% | 11.34% | 0.05% | 24.62% |

### Example 25: Formulation of the Compositions

Comp-26: A blend of Gum acacia (180 g) and water was stirred at room temperature for 15-30 minutes, followed by the addition of enriched water extract, AR-3 (640 g). Stirring continued at room temperature for 5-10 minutes. Subsequently, spent 90% ethanol extract, AR-4 (160 g) was gradually introduced at room temperature and stirred for approximately 30 minutes until achieving a homogeneous mixture. The resulting contents were then dried under reduced pressure to yield flakes. These flakes were uniformly blended with colloidal silicon dioxide (20 g) in a polyethylene bag or suitable blender. Further, pulverization and sieving through a #40 mesh produced the composition as a fine powder (Comp-26)
Comp-27: A blend of Gum acacia (1.26 Kg) and water was stirred at room temperature for 15-30 minutes, followed by the addition of AR-1 (3.59 Kg). Stirring continued at room temperature for 5-10 minutes. Subsequently, AR-11 (0.64 Kg) and AR-4 (1.37 Kg) were gradually introduced at room temperature and stirred for approximately 30 minutes until achieving a homogeneous mixture. The resulting contents were then dried under reduced pressure to yield flakes. These flakes were uniformly blended with colloidal silicon dioxide (0.14 Kg) in a polyethylene bag or suitable blender. Further pulverization and sieving through a #40 mesh produced the composition as a fine powder (Comp-27)
Comp-28: A blend of Gum acacia (1.08 Kg) and water was stirred at room temperature for 15-30 minutes, followed by the addition of AR-11 (1.44 Kg). Stirring continued at room temperature for 5-10 minutes. Then, AR-4 (3.36 Kg) was slowly added at room temperature and stirred for approximately 30 minutes until obtaining a homogeneous mixture. The resulting contents were dried under reduced pressure to yield flakes. These flakes were uniformly blended with colloidal silicon dioxide (0.12 Kg) in a polyethylene bag or suitable blender. Further pulverization and sieving through a #40 mesh produced the composition as a fine powder (Comp-28)
Comp-29: A blend of Gum acacia (0.12 Kg) and water was stirred at room temperature for 15-30 minutes, followed by the addition of AR-11 (1.1775 Kg). Stirring continued at room temperature for 5-10 minutes. Then, AR-4 (0.1725 Kg) was slowly added at room temperature and stirred for approximately 30 minutes to achieve a homogeneous mixture. The resulting contents were dried under reduced pressure to yield flakes. These flakes were uniformly blended with colloidal silicon dioxide (0.030 Kg) in a polyethylene bag or suitable blender. Further pulverization and sieving through a #40 mesh produced the composition as a fine powder (Comp-29).

### Example 25: Standardization of Formulated Asparagus racemosus Root Extract Compositions

The various formulated compositions of Asparagus racemosus root extracts were standardized to Shatavarin I, Dehydroshatavarin I, shatavarin IV, and Shatavarin IX by analytical HPLC, and the results are summarized in Table 8.

TABLE 8
Estimation of four shatavarins in compositions
(26-29) by HPLC method of analysis

| | | Dehydro | | | |
|---|---|---|---|---|---|
| | Shatavarin | shatavarin | Shatavarin | Shatavarin | |
| Product | I | I | IV | IX | Total |
| C-26 | 0.58% | 2.78% | 3.62% | 0.03% | 7.01% |
| C-27 | 0.85% | 3.91% | 2.12% | 0.05% | 6.93% |
| C-28 | 2.55% | 6.92% | 7.16% | 0.17% | 16.80% |
| C-29 | 6.08 | 7.87% | 6.01% | 1.05% | 21.01% |

### Example 26: Evaluation of Estrogen-Receptor Alpha (ER-α) Phosphorylation by Western Blot Assay

Human MCF-7 cells (ATCC Cat #HTB-22; 5×10⁵ cells/well in 3 mL) were seeded in 6-well cell culture plates and maintained in DMEM medium containing 10% FBS at 37 °C in a humidified atmosphere of 5% CO₂. After 48 hours of seeding, cells were washed twice with serum-free DMEM medium (without phenol red) and maintained in the same media for another 24 hours. Cells were treated with different test samples (0.2 μg/mL) in serum-free DMEM medium and incubated for 4 hours at 37 °C in a CO₂ incubator. The untreated cells served as a vehicle control.

Western blot: After the incubation, cell culture plates were placed on an ice tray and washed twice with cold 1×PBS. Eighty microliters of lysis buffer (10 mM Tris-HCl pH 7.4, 150 mM NaCl, 1 mM EDTA, 1 mM PMSF, 10 μg/mL Aprotinin, 10 μg/mL Leupeptin, 1% Triton X-100, 1 mM NaF, 1 mM Na₃VO₄, 0.5% Sodium deoxycholate, and 1 μM Pepstatin) was added to each well and sonicated for 30 seconds. Following homogenization, samples were centrifuged at 16128×g for 10 minutes at 4 °C to sediment unbroken cells and nuclei. Supernatant was collected, and protein was quantified using a Pierce BCA protein assay kit (Thermo Scientific Cat #23225). SDS-PAGE was performed for the protein samples, and resolved proteins were transferred onto nitrocellulose membrane using the wet blotting method. Briefly, 10 μg of protein was loaded onto acrylamide gel (7.5% resolving) and ran at 100V for approximately 1 hour 40 minutes. At the end of the run, proteins were transferred to the nitrocellulose membrane by placing transfer system at 4 °C chamber (100V for 2 hours). After the transfer, the membrane was washed in 0.1M Tris-buffered saline containing 0.05% Tween20 (TBST) and blocked in Superblock blocking buffer (Thermo Scientific Cat #37535) with constant agitation for 30 minutes. The membrane was incubated with primary antibodies recognizing phospho-ER-α (Phosphorylation site: Ser118) in TBST for 16 hours at 4 °C with agitation. The membrane was washed three times and incubated with peroxidase affiniPure goat anti-rabbit secondary antibody (Jackson ImmunoResearch Cat #111-035-045; 1:10000 dilution) for 1 hour at room temperature with agitation, followed by three times of washing in TBST for 5 minutes each. Immunoreactive bands were detected using a chemiluminescent substrate (Thermo Scientific Cat #34080) and images were captured using Bio-Rad Molecular imager (Model: ChemiDOC MP). Membrane was stripped of the phospho-ER-α antibody and re-probed with anti-3-actin antibody (Sigma Chemical Co., USA; 1:10000 dilution) by incubating at room temperature for 2 hours and images were captured. The intensities of phospho-ER-α and actin protein bands were calculated using Carestream MI software and normalized. The normalized pER-α expression (in arbitrary units) was measured using the following formula:

$$Normalized pER - α expression = \frac{phospho - ER - α expression}{β - actin expression}$$

The results of Normalized pER-α expression in the individual extracts and their compositions-treated cells are summarized in Tables 9-11.

TABLE 9
Normalized pER-α expressions in the cells treated with compositions
of Asparagus racemosus root containing water extract enriched with
90% aq. ethanol (AR-3) and spent with 90% aq. ethanol extract (AR-4)

| | | | | | | | Normalized pER-α | |
|---|---|---|---|---|---|---|---|---|
| | AR-3 | | AR-4 | | | Comp | expression | |
| Comp | | pER-α | | pER-α | | Dose | Additive | |
| # | μg/mL | expression | μg/mL | expression | Ratio | μg/mL | (Calculated) | Observed |
| C-1 | 0.16 | 0.42 | 0.04 | 0.09 | 4:1 | 0.2 | 0.51 | 0.72 |
| C-2 | 0.15 | 0.39 | 0.05 | 0.11 | 3:1 | 0.2 | 0.50 | 0.68 |
| C-3 | 0.13 | 0.34 | 0.07 | 0.15 | 2:1 | 0.2 | 0.49 | 0.69 |
| C-4 | 0.1 | 0.26 | 0.1 | 0.22 | 1:1 | 0.2 | 0.48 | 0.65 |
| C-5 | 0.07 | 0.18 | 0.13 | 0.29 | 1:2 | 0.2 | 0.47 | 0.63 |

TABLE 10
Normalized pER-α expression of compositions of Asparagus racemosus root containing
water extract enriched with 90% aq. butanol (AR-5) and spent 90% aq. ethanol extract
(AR-4); and compositions of Asparagus racemosus root containing water extract enriched
through macroporous resin (AR-11) and spent 90% aq. ethanol extract (AR-4)

| | | | | | | | Normalized pER-α | |
|---|---|---|---|---|---|---|---|---|
| | AR-5 | | AR-4 | | | Comp | expression | |
| Comp | | pER-α | | pER-α | | Dose | Additive | |
| # | μg/mL | expression | μg/mL | expression | Ratio | μg/mL | (Calculated) | Observed |
| C-6 | 0.13 | 0.32 | 0.07 | 0.15 | 2:1 | 0.2 | 0.47 | 0.69 |
| C-7 | 0.1 | 0.25 | 0.1 | 0.22 | 1:1 | 0.2 | 0.47 | 0.58 |
| C-8 | 0.07 | 0.17 | 0.13 | 0.29 | 1:2 | 0.2 | 0.46 | 0.59 |
| | AR-11 | | AR-4 | | | | | |
| C-14 | 0.13 | 0.35 | 0.07 | 0.15 | 2:1 | 0.2 | 0.51 | 0.73 |
| C-15 | 0.1 | 0.27 | 0.1 | 0.22 | 1:1 | 0.2 | 0.49 | 0.70 |
| C-16 | 0.07 | 0.19 | 0.13 | 0.29 | 1:2 | 0.2 | 0.48 | 0.69 |

TABLE 11
Normalized pER-α expression of compositions of Asparagus racemosus
root water containing water extract (AR-1), water extract enriched through
macroporous resin (AR-11), and spent 90% aq. Ethanol extract (AR-10)

| | | | | | | | | | Normalized pER-α | |
|---|---|---|---|---|---|---|---|---|---|---|
| | | | | | | | | Comp | expression | |
| | AR-1 | | AR-11 | | AR-10 | | | Dose | Additive | |
| C # | μg/Ml | pER-α | μg/mL | pER-α | μg/mL | pER-α | Ratio | μg/mL | (Calculated) | Observed |
| C-23 | 0.15 | 0.37 | 0.02 | 0.05 | 0.04 | 0.09 | 8:1:2 | 0.2 | 0.42 | 0.75 |
| C-24 | 0.07 | 0.16 | 0.07 | 0.19 | 0.07 | 0.15 | 1:1:1 | 0.2 | 0.35 | 0.73 |
| C-25 | 0.03 | 0.07 | 0.09 | 0.24 | 0.09 | 0.20 | 1:3:3 | 0.2 | 0.32 | 0.77 |

### Example 27: In Vitro Cell Based Assay for Prostaglandin E₂ (PGE₂) Inhibition

Human blood was collected from healthy volunteers from a peripheral vein using a syringe containing 2 mM EDTA. Plasma was separated by centrifugation at 1000 rpm for 10 minutes, and the residual blood was diluted with RPMI medium supplemented with 10% FBS and 2 mM EDTA in a ratio of 1:3. Thirty millilitres of blood was carefully layered onto the 15 mL of Ficoll/Lymphoprep in a 50 mL falcon tube in dark and tubes were centrifuged at 350×g for 30 minutes at an acceleration of 9 without using a brake. Buffy coat (interface between medium and Ficoll) containing peripheral blood mononuclear cells (PBMC) was collected carefully in 25 mL of cold 1× phosphate-buffered saline (PBS) and centrifuged at 1200 rpm for 10 minutes. Residual RBCs found in PBMCs pellet were removed by treating with ACK lysis buffer (Gibco Cat #A10492-01) and washed with fresh 1×PBS. PBMC were seeded in a 96-well plate with a density of 0.1×10⁶ cells/well and treated with different concentrations of test samples. Cells with 0.2% DMSO served as vehicle control. The plate was incubated in a CO₂ incubator at 37 °C for 2 hrs. Finally, cells were induced with LPS (10 ng/mL final concentration) for 4 hours except for vehicle control by keeping the plate at 37 °C in a CO₂ incubator. Cells treated only with LPS served as induction control. Plate was centrifuged at 1200 rpm for 5 minutes, and 120 μL cell-free supernatants were collected. Quantitation of PGE₂ was performed using an ELISA kit (Cayman Chemicals Cat #514010) according to the manufacturer's instructions. Absorbance was measured at 412 nm in a kinetic mode for 30 minutes in a Spectramax 2e plate reader. The inhibition of PGE₂ was calculated using the following formula.

% Inhibition of PGE₂=[(PGE₂ conc. in Induction)−(PGE₂ conc. in Test sample)]/(PGE₂ conc. in Induction)×100

The percentage inhibition of PGE₂ production of the individual extracts and their compositions are summarized in Tables 12-15.

TABLE 12
PGE₂ inhibition by the Asparagus racemosus
water extract and inventive composition

| | | % Inhibition of | % Improvement |
|---|---|---|---|
| | Product | PGE₂ production at | from regular |
| Exam # | code | 10 μg/mL | extract (AR) |
| Comp | AR-1 | 15.23 | — |
| ex 1 | | | |
| Ex 13 | Comp-1 | 39.71 | 24.48 |

TABLE 13
Inhibition of PGE₂ by the compositions of Asparagus racemosus root containing water
extract enriched with 90% aq. ethanol (AR-3) and spent 90% aq. ethanol extract (AR-4)

| | | | | | | | % Inhibition of | |
|---|---|---|---|---|---|---|---|---|
| | AR-3 | | AR-4 | | | Comp | PGE₂ production | |
| Comp | | PGE₂ | | PGE₂ | | Dose | Additive | |
| # | μg/mL | inhibition | μg/mL | inhibition | Ratio | μg/mL | (Calculated) | Observed |
| C-1 | 8 | 18.66 | 2 | 5.94 | 4:1 | 10 | 24.60 | 39.71 |
| C-2 | 7.5 | 17.49 | 2.5 | 7.43 | 3:1 | 10 | 24.92 | 37.59 |
| C-3 | 6.67 | 15.55 | 3.33 | 9.89 | 2:1 | 10 | 25.45 | 40.17 |
| C-4 | 5 | 11.66 | 5 | 14.86 | 1:1 | 10 | 26.52 | 41.26 |
| C-5 | 3.33 | 7.77 | 6.67 | 19.82 | 1:2 | 10 | 27.58 | 38.59 |

TABLE 14
Inhibition of PGE₂ by the compositions of Asparagus racemosus root containing water
extract enriched with 90% aq. butanol (AR-5) and spent 90% aq. ethanol extract (AR-4);
and compositions of Asparagus racemosus root containing water extract enriched
through macroporous resin (AR-11) and spent 90% aq. ethanol extract (AR-4)

| | | | | | | | % Inhibition of | |
|---|---|---|---|---|---|---|---|---|
| | AR-5 | | AR-4 | | | Comp | PGE₂ production | |
| Comp | | PGE₂ | | PGE₂ | | Dose | Additive | |
| # | μg/mL | inhibition | μg/mL | inhibition | Ratio | μg/mL | (Calculated) | Observed |
| C-6 | 6.67 | 16.62 | 3.33 | 9.89 | 2:1 | 10 | 26.52 | 44.25 |
| C-7 | 5 | 12.46 | 5 | 14.86 | 1:1 | 10 | 27.32 | 39.72 |
| C-8 | 3.33 | 8.30 | 6.67 | 19.82 | 1:2 | 10 | 28.11 | 42.43 |
| | AR-11 | | AR-4 | | | | | |
| C-14 | 6.67 | 19.29 | 3.33 | 9.89 | 2:1 | 10 | 29.18 | 49.50 |
| C-15 | 5 | 14.46 | 5 | 14.86 | 1:1 | 10 | 29.32 | 47.29 |
| C-16 | 3.33 | 9.63 | 6.67 | 19.82 | 1:2 | 10 | 29.45 | 45.87 |

TABLE 15
Inhibition of PGE₂ of the compositions of Asparagus racemosus root
containing water extract (AR-1), water extract enriched through macroporous
resin (AR-11), and spent 90% aq. ethanol extract (AR-10)

| | | | | | | | | | % Inhibition of | |
|---|---|---|---|---|---|---|---|---|---|---|
| | | | | | | | | Comp | PGE₂ production | |
| | AR-1 | | AR-11 | | AR-10 | | | Dose | Additive | |
| C # | μg/mL | PGE₂ | μg/mL | PGE₂ | μg/mL | PGE₂ | Ratio | μg/mL | (Calculated) | Observed |
| C-23 | 7.27 | 11.07 | 0.91 | 2.63 | 1.82 | 5.41 | 8:1:2 | 10 | 19.11 | 39.46 |
| C-24 | 3.33 | 5.07 | 3.33 | 9.63 | 3.33 | 9.89 | 1:1:1 | 10 | 24.60 | 43.89 |
| C-25 | 1.43 | 2.18 | 4.29 | 12.41 | 4.29 | 12.75 | 1:3:3 | 10 | 27.33 | 44.65 |

Example 28: In-vivo efficacy of AR-1 and Comp-1 in mitigating Cyclophosphamide-induced primary ovarian insufficiency in Sprague Dawley rats. Following a 7-day acclimatization period, female Sprague Dawley rats (age: 10-12 weeks; Body weight: 230-290 g) were randomly divided into four groups (n=8): vehicle control (G1), Cyclophosphamide (CYP) mg/kg (G2), AR-1 (100 mg/kg; G3) and comp-1 (100 mg/kg; G4). On day 8 of the experiment, each rat in groups G2, G3, and G4 received (i.p) 200 mg/kg CYP, followed by 8 mg/kg/day CYP from day 9 to 21. The G1 and G2 rats received 0.5% Sodium carboxymethyl cellulose (CMC-Na), and G3 and G4 rats received the test supplements (AR-1 and comp-1) daily through oral gavage for 21 consecutive days. On Day 22, all animals were sacrificed using CO₂ inhalation. Ovaries were collected, fixed in 10% formalin, and embedded in paraffin wax. The paraffin-embedded tissue specimens were cut into m thick sections, mounted on clean glass slides and stained with hematoxylin and eosin (H-E). The stained tissue sections were examined under the 20× objective of a light microscope (Axio Scope A1, Carl-Zeiss, Germany). Twenty randomly selected fields were analysed for primordial, primary, antral, griffin, and atretic follicles, and the average of total number of follicles present in each tissue section was calculated. The data is presented in Table 16.

For example, the Asparagus racemosus water extract (AR-1) and Asparagus racemosus enriched extract composition (comp-1), each at 100 mg dose, showed 32.68% and 51.15% improvements in total follicular count, respectively, over the Cyclophosphamide control (G2). Surprisingly, the enriched Asparagus racemosus extract composition showed higher efficacy (Table-16). Reduced number of ovarian follicles is a major hallmark of primary ovarian insufficiency, also known as premature ovarian failure, that causes hormonal imbalance, disturbs the regular menstrual cycle, infertility, and menopause-associated symptoms.

TABLE 16
Evaluation of Asparagus racemosus enriched extracts supplementation
on total follicular count of female Sprague Dawley rats

| | | Average total | % increase |
|---|---|---|---|
| | | follicular count | over CYP |
| Animal groups | Dose | (per section) | control |
| G1: Vehicle control | — | 13.81 | |
| G2: Cyclophosphamide | — | 5.63 | |
| (CYP) control | | | |
| G3: AR-1 | 100 mg/Kg | 7.47 | 32.68 |
| G4: Comp-1 | 100 mg/Kg | 8.51 | 51.15 |

Note:
Higher the values are better efficacy

### Example 29: Efficacy of Comp-26, Comp-27, and Comp-28 Supplementation in Comparison with 90% Aqueous Ethanol Extract of Shatavari (AR-2) in Letrozole-Induced PCOS in Female Sprague Dawley Rats

Following a 7-day acclimatization period, female Sprague Dawley rats (age: 10-12 weeks, body weight 250-300 g) were randomly assigned to six groups (n=7): vehicle control (G1), Letrozole (G2; 1 mg/kg), AR-2 (G3; 20 mg/kg), Comp-26 (G4; 20 mg/kg), Comp-27 (G5; 20 mg/kg), and Comp-28 (G6; 20 mg/kg).

The study was performed in two phases i.e., the induction and supplementation phases. In the induction phase, all the animals, except for G1, were gavaged with letrozole to induce PCOS for 21 days. In the supplementation phase (from days 22 to 48), all rats were supplemented once daily with either the vehicle or one of the test items. An oral glucose tolerance test (OGTT) was performed on day 49 in overnight fasted rats. Blood samples were collected on day 48. The serum was separated by centrifuging the blood samples at 3200×g for 15 minutes at 4 °C and stored at −80 °C in aliquots for biomarker analysis. On day 50, all rats were euthanized using CO₂ asphyxiation, and their ovaries were collected. The ovaries were fixed in 10% normal buffered formalin for histopathology examination. The fixed tissues were paraffin-embedded, sectioned (5-7 μm) using a microtome, and stained with hematoxylin-eosin following the standard protocols. The stained tissue sections were examined at 10× magnification under a light microscope (Carl Zeiss, Germany).

Results of oral glucose tolerance test (OGTT), serum hormone levels such as 17β-estradiol and testosterone are presented in Table 17 and 18. Furthermore, total follicles, Graafian follicles, and Follicular cysts are presented in Table 19.

TABLE 17
Evaluation of Comp-26, Comp-27, and Comp-28 supplementation
in comparison with 90% aqueous ethanol extract
of Shatavari (AR-2) on oral glucose tolerance test
(OGTT) of female Sprague Dawley rats

| | | % improvement |
|---|---|---|
| Groups | AUC (mg/dL/min) | from G2 |
| G1 - Vehicle Control | 14100 ± 2769 | — |
| G2-Letrozole (1 mg/kg) | 21030 ± 4007 | — |
| G3-AR-2 (20 mg/kg) | 18990 ± 1589 | 9.70 |
| G4-Comp-26 (20 mg/kg) | 18189 ± 576 | 13.51 |
| G5-Comp-27 (20 mg/kg) | 17534 ± 953 | 16.62 |
| G5-Comp-28 (20 mg/kg) | 16813 ± 1200 | 20.05 |

Values were expressed as Mean ± SD (n = 7).

TABLE 18
Evaluation of Comp-26, Comp-27, and Comp-28 supplementation
in comparison with 90% aqueous ethanol extract of Shatavari
(AR-2) on serum hormones levels such as 17β-estradiol
and testosterone of female Sprague Dawley rats

| | 17β-Estradiol | | Testosterone | |
|---|---|---|---|---|
| | | % | | % |
| | | improve- | | improve- |
| | Concentration | ment | Concentration | ment |
| Groups | (pg/mL) | from G2 | (pg/ml) | from G2 |
| G1 - Vehicle | 41.35 ± 23.93 | — | 0.26 ± 0.10 | — |
| Control | | | | |
| G2-Letrozole | 32.97 ± 13.89 | — | 3.18 ± 1.16 | — |
| (1 mg/kg) | | | | |
| G3-AR-2 | 35.36 ± 18.83 | 7.24 | 2.21 ± 0.87 | 30.50 |
| (20 mg/kg) | | | | |
| G4-Comp-26 | 36.51 ± 18.83 | 10.74 | 1.81 ± 0.96 | 43.08 |
| (20 mg/kg) | | | | |
| G5-Comp-27 | 37.57 ± 16.00 | 13.95 | 1.26 ± 0.41 | 60.38 |
| (20 mg/kg) | | | | |
| G6-Comp-28 | 39.30 ± 12.10 | 19.20 | 0.94 ± 0.72 | 70.44 |
| (20 mg/kg | | | | |

Values were expressed as Mean ± SD (n = 7).

TABLE 19
Evaluation of Comp-26, Comp-27, and Comp-28 supplementation in comparison
with 90% aqueous ethanol extract of Shatavari (AR-2) on total follicles,
Graafian follicle, and follicular cysts of female Sprague Dawley rats

| | Total Follicles | | Graafian follicle | | Follicular cysts | |
|---|---|---|---|---|---|---|
| | count | % | count | % | count | % |
| | (mean ± | improvements | (mean ± | improvements | (mean ± | improvements |
| Groups | SD) | from G2 | SD) | from G2 | SD) | from G2 |
| G1 - Vehicle | 17.14 ± 4.74 | — | 4.23 ± 1.19 | — | 2.29 ± 0.76 | — |
| Control | | | | | | |
| G2-Letrozole | 10.89 ± 5.84 | — | 2.05 ± 0.89 | — | 8.33 ± 2.58 | — |
| (1 mg/kg) | | | | | | |
| G3-AR-2 (20 | 13.02 ± 4.79 | 19.56 | 2.39 ± 0.71 | 16.59 | 6.05 ± 1.15 | 27.38 |
| mg/kg) | | | | | | |
| G4-Comp-26 | 14.23 ± 6.06 | 30.67 | 2.63 ± 1.05 | 28.29 | 5.16 ± 1.91 | 38.06 |
| (20 mg/kg) | | | | | | |
| G5-Comp-27 | 16.00 ± 4.36 | 46.92 | 2.83 ± 0.94 | 38.05 | 4.43 ± 2.15 | 46.82 |
| (20 mg/kg) | | | | | | |
| G6-Comp-28 | 16.29 ± 4.89 | 49.59 | 3.5 ± 0.84 | 70.73 | 4.11 ± 1.50 | 50.66 |
| (20 mg/kg) | | | | | | |

Values were expressed as Mean ± SD (n = 7).

It will be understood that the above description is intended to be illustrative and not restrictive. The embodiments will be apparent to those in the art upon reviewing the above description. The scope of the invention should therefore, be determined not with reference to the above description but should instead be determined by the appended claims along with full scope of equivalents to which such claims are entitled.

1. A synergistic enriched Asparagus racemosus extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; for female health benefits.

2. The synergistic enriched extract compositions as claimed in claim 1, wherein the compositions contain optionally at least one additional component selected from the group consisting of pharmaceutically or nutraceutically, or dietically acceptable excipients, carriers, and diluents; selected from monosaccharides, disaccharides, polysaccharides, dextrins, polyhydric alcohols or sugar alcohols, cellulose-based derivatives, silicates, metallic stearates, organic acids, fatty acid esters, calcium pantothenate, amino acids, proteins, organic metal salts, natural pigments, flavors, Class I & Class II preservatives and mixtures thereof.

3. The synergistic enriched extract compositions as claimed in claim 2, wherein the monosaccharides are selected from glucose, dextrose, fructose, galactose; disaccharides are selected sucrose, maltose, lactose, lactulose, trehalose cellobiose, chitobiose; polysaccharides are selected from starch and modified starch such as sodium starch glycolate, pre-gelatinized starch, soluble starch, ultrasperse A and other modified starches; dextrins are selected from yellow dextrin, white dextrin, maltodextrin, glucidex 12D, rice maltodextrin, tapioca/cassava maltodextrin; polyhydric alcohols or sugar alcohols are selected sorbitol, mannitol, inositol, xylitol, isomalt; cellulose based derivatives are selected from microcrystalline cellulose, hydroxy propyl methyl cellulose, hydroxy ethyl cellulose; silicates are selected neusilin, veegum, talc, colloidal silicon dioxide, syloids; metallic stearates are selected from calcium stearate, magnesium stearate, zinc stearate; organic acids are selected from citric acid, tartaric acid, malic acid, succinic acid, lactic acid, L-ascorbic acid; natural gums are selected acacia, carrageenan, guar gum, xanthan gum, gum ghatti etc., bio polymers/bio polysaccharides such as sodium alginate, calcium alginate, and alginic acid; proteins are selected from whey protein, whey isolate, casein, gelatin, pectin, and agar; organic metal salts are selected from sodium chloride, calcium chloride, dicalcium phosphate, zinc sulphate, zinc chloride; natural sweeteners such as licorice, Stevia, steviosides such as rebaudioside, palm sugars, and natural sweet proteins such as brazzein, monelin, curculin, mabinlin, miraculin, and pentadin, etc.

4. The synergistic enriched extract compositions as claimed in claim 2,
wherein the enriched Asparagus racemosus extract composition varies in the range of 90%-10% by weight and excipients in the composition varies in the range of 10%-90% by weight.

5. The synergistic enriched extract compositions as claimed in claim 1, wherein the enriched Asparagus racemosus extract composition optionally contains at least one additional ingredient selected from the group consisting of shatavarin II, shatavarin III, shatavarin V, shatavarin VI, shatavarin VII, shatavarin VIII, shatavarin IX, asparacoside, asparanin and immunoside or mixtures thereof.

6. The synergistic enriched extract compositions as claimed in claim 1, wherein the enriched Asparagus racemosus extract composition; optionally contains at least one extract or fraction selected from the group consisting of Nigella sativa, Cinnamomum zeylanicum, Foeniculum vulgare, Trigonella foenum-graecum, Glycyrrhiza glabra and Linum usitatissimum or atleast one phytochemical selected from Inositols such as myo-inositol or D-chiro-inositol or mixtures thereof.

7. A synergistic enriched Asparagus racemosus extract composition comprising: (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; for female health benefits; wherein the said composition is prepared by a process comprising the steps of, (a) enriching water extract of Asparagus racemosus (Shatavari) root by re-extracting with a polar organic solvent or by loading water extract on macroporous resin and eluting with an aqueous alcohol to obtain enriched extract; (b) extracting spent or residual Asparagus racemosus (Shatavari) root with polar organic solvent to obtain second extract; (c) combining the extracts obtained from step (a) and step (b) to obtain enriched Asparagus racemosus composition; and d) optionally blending the enriched Asparagus racemosus composition with at least one component selected from pharmaceutically or nutraceutically or dietically acceptable excipients, carriers and diluents.

8. The process for preparing synergistic enriched Asparagus racemosus extract composition as claimed in claim 7 comprises the steps of,
(a) extracting Asparagus racemosus root powder with water,
(b) evaporating the water extract to a minimum volume to obtain the concentrate,
(c) re-extracting the step (b) concentrate with a polar organic solvent,
(d) evaporating the polar organic solvent soluble extract to give an enriched extract,
(e) alternately passing the water extract of step (a) on adsorbent resin and eluting with aqueous alcohol,
(f) evaporating the aqueous alcohol eluent to give an enriched extract,
(g) extracting the Asparagus racemosus root spent residue from step (a) with a polar organic solvent,
(h) evaporating the polar organic solvent extract in step (g) to give a second extract,
(i) combining the extracts of step (d) or step (f) and step (h) to obtain synergistic enriched Asparagus racemosus extract composition; and
(j) optionally blending Asparagus racemosus synergistic enriched extract composition of step (i) with at least one component selected from pharmaceutically or nutraceutically or dietically acceptable excipients, carriers and diluents.

9. The process as claimed in claim 8, wherein the polar organic solvent is selected from ethanol, n-butanol, methanol, acetone, aqueous ethanol, aqueous methanol, aqueous acetone, and mixtures thereof and the aqueous alcohol is selected from methanol, ethanol, and n-butanol.

10. The synergistic enriched Asparagus racemosus extract compositions as claimed in claim 2, wherein the composition is formulated into a dosage form selected from dry powder form, liquid form, beverage, food product, dietary supplement, or any suitable form such as a tablet, a capsule, a soft chewable tablet, gummies, or gummy bar.

11. The synergistic enriched Asparagus racemosus extract compositions as claimed in claim 2, wherein the composition is formulated into a nutritional/dietary supplement that can be contemplated/made into the dosage form of healthy foods or food for specified health uses, such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

12. The synergistic enriched Asparagus racemosus extract compositions as claimed in claim 2, wherein the composition is formulated into a controlled-release tablet, using controlled-release polymer-based coatings by techniques including nanotechnology, microencapsulation, colloidal carrier systems, and other drug delivery systems for obtaining the desired therapeutic benefit.

13. A method of obtaining at least one female reproductive and/or sexual health benefit selected from promoting or regulating the production of reproductive hormones to alleviate the hormonal deficiency and/or hormonal imbalance, reducing symptoms of perimenopause, premenstrual syndromes (PMS), menopausal disorders including discomforts related to the menstrual cycle, alleviating the symptoms of polycystic ovary syndrome (PCOS), Polycystic Ovarian Disease (PCOD), gynecological disorders, and urogenital or reproductive system infections; supporting ovarian function, and maintaining libido, reducing coital pain and improving lactation; wherein the method comprises supplementing the subject in need thereof with an effective dose of a Asparagus racemosus synergistic enriched extract composition comprising; (i) shatavarin I in the range of 0.5-15%; (ii) dehydroshatavarin I in the range of 0.1-25%; (iii) shatavarin IV in the range of 2-25% and (iv) shatavarin IX in the range of 0.001-1.0%; and (v) optionally with at least one component selected from pharmaceutically or nutraceutically or dietically acceptable excipients, carriers, and diluents.

14. (canceled)

15. Dehydroshatavarin I of the formula: 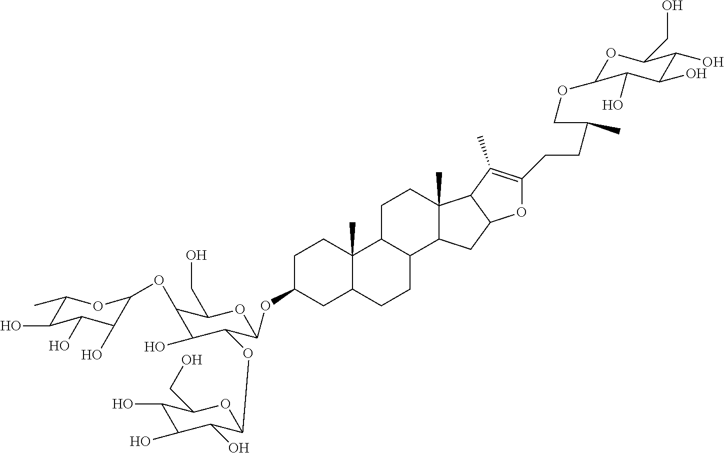
Characterized by ¹H NMR (d₆-DMSO): δ 5.16 (1H, d, J=4.4 Hz), 5.13 (1H, d, J=4.0 Hz), 4.90 (1H, d, J=4.8 Hz), 4.81-4.85 (4H, m), 4.72 (1H, s), 4.62-4.68 (4H, m), 4.59 (1H, d, J=5.2 Hz), 4.47 (1H, d, J=8.0 Hz), 4.41 (1H, d, J=5.6 Hz), 4.37 (1H, t, J=6.4 Hz), 4.33 (1H, d, J=7.6 Hz), 4.27-4.32 (1H, m), 4.16 (1H, t, J=5.6 Hz), 4.08 (1H, d, J=8.0 Hz), 3.64-3.67 (3H, m), 3.61 (1H, brs), 3.57-3.58 (1H, m), 3.37-3.51 (7H, m), 3.03-3.19 (9H, m), 2.92-3.01 (2H, m), 2.01-2.13 (3H, m), 1.63-1.84 (5H, m), 1.55 (3H, s), 1.36-1.52 (9H, m), 1.15-1.28 (5H, m), 1.10 (3H, d, J=6.4 Hz), 1.06-1.09 (2H, m), −0.95-1.05 (2H, m), 0.90 (3H, s), 0.89 (3H, d, J=6.4 Hz), 0.62 (3H, s). Mass (Q-tof, negative ion mode): m/z 1047.5400 (M−H)⁻.

16. The Dehydroshatavarin I as claimed in claim 15 obtained from the root extract of Asparagus racemosus.
